SEQUENCE LISTING (19 sequences)

SEQ ID NO: 1 - protein, 12 aa, Homo sapiens
ENEGFTVTAEGK

SEQ ID NO: 2 - protein, 17 aa, Homo sapiens
SEETKENEGFTVTAEGK

SEQ ID NO: 3 - protein, 12 aa, Homo sapiens
SGSDEVQVGQQR

SEQ ID NO: 4 - protein, 11 aa, Homo sapiens
ACEPGVDYVYK

SEQ ID NO: 5 - protein, 11 aa, Homo sapiens
VTIKPAPETEK

SEQ ID NO: 6 - protein, 10 aa, Homo sapiens
SDDKVTLEER

SEQ ID NO: 7 - protein, 12 aa, Homo sapiens
VSHSEDDCLAFK

SEQ ID NO: 8 - protein, 8 aa, Homo sapiens
EALKLEEK

SEQ ID NO: 9 - protein, 10 aa, Homo sapiens
CAEENCFIQK

SEQ ID NO: 10 - protein, 16 aa, Homo sapiens
VHQYFNVELIQPGAVK

SEQ ID NO: 11 - protein, 9 aa, Homo sapiens
NTLIIYLDK

SEQ ID NO: 12 - protein, 14 aa, Homo sapiens
GQGTLSVVTMYHAK

SEQ ID NO: 13 - protein, 10 aa, Homo sapiens
FYHPEKEDGK

SEQ ID NO: 14 - protein, 9 aa, Homo sapiens
AKDQLTCNK

SEQ ID NO: 15 - protein, 7 aa, Homo sapiens
TFISPIK

SEQ ID NO: 16 - protein, 9 aa, Homo sapiens
YISKYELDK

SEQ ID NO: 17 - protein, 6 aa, Homo sapiens
FYHPEK

SEQ ID NO: 18 - protein, 12 aa, Homo sapiens
ELISNASDALDK

SEQ ID NO: 19 - protein, 10 aa, Homo sapiens
EDQTEYLEER

(19) United States
(12) Patent Application Publication
(10) Pub. No.: US 2017/0219579 A1
(43) Pub. Date: Aug. 3, 2017

(54) ANTISECRETORY FACTOR COMPLEX ASSAY

(71) Applicant: LANTMÄNNEN AS-FAKTOR AB

(72) Inventors: Stefan Lange, Göteborg (SE); Ivar Lönnroth, Mölndal (SE)

(73) Assignee: LANTMÄNNEN AS-FAKTOR AB, Stockholm (SE)

(21) Appl. No.: 15/314,158

(22) PCT Filed: May 28, 2015
(86) PCT No.: PCT/EP2015/061899

§ 371 Date: Nov. 28, 2016

(30) Foreign Application Priority Data
May 28, 2014 (SE) 1450640-6

(51) Int. Cl. G01N 33/564

(52) U.S. Cl. CPC G01N 33/564; G01N 2333/4716; G01N 2800/7095

(57) ABSTRACT

The present invention relates to animmunological assay kits for determining the presence or absence, and/or the concentration, of proteasome-complement complex formation in a sample, such as in bodily fluids using a first antibody and a second antibody, wherein the first antibody is immobilized on a carrier and the second antibody is modified with a labeling substance, and the first antibody and the second antibody are selected from an antibody specific for a proteasome protein, such as AF1 or intact proteasome, and an antibody specific for complement factor C3, such as C3, C3c,C3b, iC3b, or an antibody specific for complement factor C4, such as C4, C4b, iC4b or C4c

The disclosed assay can be used for detecting levels of circulating 26S proteasome bound to complement factor 3 or 4 in blood plasma or other body fluids, such as for monitoring levels of inflammation and virus infection in the body of a mammalian, including complement system down regulation in the body of a mammalian as well as for verifying compliance of processed cereals (SPC) and/or functional food with high levels of natural antisecretory protein (NASP).

### FIELD OF INVENTION

Antisecretory factor (AF) is a protein complex which inhibits inflammation and regulates fluid transport. The present invention relates to the surprising discovery that the AF complex resides in modified proteasomes and complement C3 and/or C4. In addition, for the first time, a virus induced complex between proteasomes and complement factor C3 and/or C4 is described. Proteasome-complement C3 and/or C4 complexes are herein for the first time purified from human blood and analyzed by western blot and mass-spectroscopy. Antibodies specific for proteasome subunits are disclosed herein and their use in novel immunoassays. In one embodiment, an ELISA test discloses the binding of AF-exposing proteasomes to complement factor C3c, C3b, iC3b, C4b, iC4b or C4c, in another embodiment, an ELISA test discloses the binding of intact proteasomes to complement factor C3, C3c, C3b, iC3b, C4, C4b, iC4b or C4c. The herein described assays measure AF, which is increased about tenfold following intake of processed cereals (SPC). No difference in the levels of complement factors H and I were seen. The proteasome subunits are shown to be partially split, exposing a prior hidden antisecretory peptide sequence. Also C3 is partially split into its inactive form C3c upon proteasome binding, leading to an anti-inflammatory effect during AF complex formation.

An immunological assay kit is thus disclosed for determining the presence or absence, and/or the concentration, of AF-C3 complex formation in bodily fluids, using a first antibody and a second antibody, wherein the first antibody is immobilized on a carrier and the second antibody is modified with a labeling substance.

For measuring AF, the first antibody and the second antibody are selected from an antibody specific for AF1 and an antibody specific for complement factor C3, such as C3c.

In order to measure a general complex-formation between proteasomes and complement factor C3 or C4, a first antibody and the second antibody are selected from an antibody against proteasomes and against complement factor C3 or C4.

The disclosed assay can be used for monitoring levels of inflammation in the body of a mammalian including measuring and/or monitoring complement system down regulation in the body of a mammalian, as well as for verifying compliance of a human or animal to processed cereals (SPC) and/or functional food with high levels of natural antisecretory protein (NASP), as well as for measuring and/or monitoring impact of virus on complement system regulation in body fluids.

### BACKGROUND OF THE INVENTION

The Complement System

The complement system plays a major role in defense against pathogens. It also identifies dying cells, immune complexes or misfolded molecules and guides adaptive immunity. The physiological relevance of complement is demonstrated by illnesses affecting complement deficient patients such as recurrent infections, autoimmune diseases and kidney diseases.

Invading pathogens activate complement either spontaneously, due to differences in surface composition compared to host cells, or through antibody or pentraxin binding. This leads to rapid initiation of a proteolytic complement cascade, release of pro-inflammatory anaphylatoxins that influence blood vessel permeability (C5a, C3a) and attract white blood cells (C5a), opsonisation of the target with C3b and finally formation of the membrane attack complex (MAC).

Complement has to be tightly regulated by both soluble and membrane bound regulators to protect self-tissues from complement-mediated damage. Many of these inhibitors are located in chromosome 1q32 and they are collectively termed regulators of complement activation (RCA). The RCA proteins inhibit the complement system by accelerating the decay of the C3 and C5 convertases and/or by acting as a cofactor for a serine proteinase factor I (FI) in the degradation of C3b and C4b. FI inhibits all the pathways of complement by cleaving the activated C3b and C4b proteins. This, however, can only occur in the presence of cofactors.

The complement system is mostly associated with the inflammatory response to bacterial infections, where the different complement factors react in sequence to finally lysate the bacterial cell wall. It is less known that the key factors C3 and C3b can be inactivated by hydrolysis to C3c, which is known to be achieved by proteolytic factor I (F1). This down-regulation is necessary to restore the complement system after inflammation, which otherwise would be turned on indefinitely.

Proteasomes

Proteasomes are present in all cells, where they are essential for the degradation and regulation of proteins. Its catalytic 20S subunit consists of 7 structural proteins and 7 proteolytic proteins, while the two regulative 19S subunits consist of 19 different proteins. Most, if not all proteasome proteins, have been identified in blood plasma in concentrations similar to those found in tissues, as described in the HUPO project gene cards (https://www.GeneCards.org). 20S proteasomes detected e.g. in human serum are generally designated as “circulating proteasomes” (c-proteasomes). The level of plasma circulating proteasomes has been shown to be elevated in certain cancer forms and autoimmune diseases.

The present inventors have previously isolated a protein named AF1 (antisecretory factor 1) from blood, and sequenced its encoding gene. Later, AF1 was shown to be a constituent of the 19S proteasome subunit, and as such named PSMD4, RPN10 or S5a. It was further shown that bacterial enterotoxins and processed cereals were able to induce an altered form of antisecretory factor (AF), which inhibited inflammation and fluid secretion in the gut. This modified form of AF was found to bind to the polysaccharide agarose. After elution with a-methylglucoside, its concentration could be determined by ELISA.

Surprisingly, it is herein for the first time demonstrated that proteasomes react with the complement factors C3 after intake of processed cereals (SPC). This reaction results in exposure of previously hidden antisecretory epitopes, which can be assayed in a double sandwich ELISA using antibodies against AF-1 and C3. Furthermore, it could herein for the first time be shown that the proteasome/complement complex formation results in the splitting of C3 into its inactive form C3c.

Antisecretory Factor (AF)

Antisecretory factor (AF) is a 41 kDa protein that originally was described to provide protection against diarrhea diseases and intestinal inflammation (for a review, see: The antisecretory factor: synthesis, anatomical and cellular distribution, and biological action in experimental and clinical studies. Int Rev Cytol, 2001. 210: p. 39-75.). The antisecretory factor (AF) protein has been sequenced and its cDNA cloned. The antisecretory activity seems to be mainly exerted by a peptide located between the amino acid positions 35 and 50 on the antisecretory factor (AF) protein sequence and comprising at least 4-16, such as 4, 6, 7, 8 or 16 amino acids of the consensus sequence. Immunochemical and immunohistochemical investigations have revealed that the antisecretory factor (AF) protein is present and may also be synthesized by most tissues and organs in a body. Synthetic peptides, comprising the antidiarrhoeic sequence, have prior been characterized (WO 97/08202; WO 05/030246). Antisecretory factor (AF) proteins and peptides have previously been disclosed to normalize pathological fluid transport and/or inflammatory reactions, such as in the intestine and the choroid plexus in the central nervous system after challenge with the cholera toxin (WO 97/08202). Food and feed with the capacity to either induce endogenous synthesis of AF or uptake of added AF have therefore been suggested to be useful for the treatment of edema, diarrhea, dehydration and inflammation e.g. in WO 97/08202. WO 98/21978 discloses the use of products having enzymatic activity for the production of a food that induces the formation of antisecretory factor (AF) proteins. WO 00/038535 further discloses food products enriched in native antisecretory factor (AF) proteins as such (NASP).

Antisecretory factor (AF) proteins and fragments thereof have also been shown to improve the repair of nervous tissue, and proliferation, apoptosis, differentiation, and/or migration of stem and progenitor cells and cells derived thereof in the treatment of conditions associated with loss and/or gain of cells (WO 05/030246) and to be equally effective in the treatment and/or prevention of intraocular hypertension (WO 07/126364), as for the treatment and/or prevention of compartment syndrome (WO 07/126363).

What is more, the present inventors have recently shown that AF is able to monitor and/or beneficially affect the structure, distribution and multiple functions of lipid rafts, receptors and/or caveolae in membranes and can thus be employed for the treatment and/or prevention of structural disorganization and dysfunction of lipid rafts and/or caveolae in cell membranes (WO 07/126365).

The present inventors have further been able to prove that the same antisecretory factor (AF) protein, as well as peptides and fragments thereof can intervene in the biological activation of transmembrane proteins, e.g. NKCC1 through FAK and CAP, and that it can thus directly regulate the pathological activity of the ion channel in pathological and/or perturbed cells, effectively normalizing the intracellular pressure and transmembrane protein function in said cell, and thus allowing an improved uptake of drugs used in e.g. cancer therapy (WO 2010/093324).

The present application for the first time sheds light on the former undocumented interaction between the complement system and proteasomes and the fact that as a result of this interaction, the antisecretory sequence of AF is exposed and possibly released into the blood. Due to this newfound insight in the natural interaction pathway between these different compounds, the inventors have for the first time been able to develop a novel and highly effective immunological assay for detecting proteasome-C3 complex-formation in bodily fluids. The new assay for the first time provides a quick and efficient tool for monitoring levels of inflammation in the body of a mammalian, including complement system regulation in the body of a mammalian, as well as for verifying compliance of human and/or animals to processed cereals (SPC) and/or functional food with high levels of natural antisecretory protein (NASP).

Herpes Simplex Virus-1

Herpes simplex encephalitis (HSE) is the most common cause of viral encephalitis in the Western world, responsible for up to 20% of the cases. Affecting 2-4 inhabitants per million people per year, HSE can be caused by either primary or recurrent infection of herpes simplex virus type 1 (HSV-1). Brain infection resulting in HSE has been suggested to occur after viral invasion via the trigeminal nerve, the olfactory tract or via both routes. It has been reported that in early stages of HSE, abundance of HSV-1 antigen in the olfactory bulb (OB) and tract might be related to the final location of inflammation and necrosis in the central nervous system (CNS). In addition, the viral spread within the brain and between the two brain hemispheres has been less extensively examined, but is most likely decisive for location and development of clinical manifestations of HSE.

In rodent models, viral spread of HSE via the olfactory tract in mice and rats has been observed to extend from the nasal cavity into the CNS. The olfactory receptor neuron passes through the olfactory epithelium, penetrates the cribriform plate and enters the OB to reach CNS. In the OB, the olfactory receptor neurons connect in glomeruli to mitral cells (second-order neurons), which in turn project to the olfactory system and the limbic system. The limbic system, which involves amygdala, hippocampus and the OBs, is evolutionary one of the oldest parts of the brain, and it has been suggested that the clinical manifestations of HSE are due to a special affinity to the limbic cortices for HSV-1 as HSE cause selective damage to the grey matter of the entire limbic system. Hence, HSV-1 may find its way from the OB through the brain using evolutionary conserved routes.

The present applications further for the first time shows that complement C3 and C4, respectively, form a complex with proteasomes after HSV-1virus infection. This reaction is estimated with a sandwich ELISA using antibodies against intact proteasomes and against C3 or C4.

### SUMMARY OF THE PRESENT INVENTION

The present invention provides an immunological assay and an immunological assay kit for detecting different proteasome-C3 and/or C4 complex-formation(s) in bodily fluids.

A novel immunological assay and/or immunological assay kit is herein disclosed which provides a quick and efficient tool for monitoring, verifying and/or detecting status of inflammation(s) in the body of a mammalian, e.g. but not limited to after and/or during ongoing virus infection, and complement system (down-) regulation in the body of said mammalian, as well as for verifying compliance of a human and/or animal to processed cereals (SPC) and/or a functional food, food supplement, feed or feed supplement with a high level of natural antisecretory protein (NASP), such as egg-yolk (Salovum©).

The present immunological assay, as well as the present immunological assay kit, uses at least a first antibody and a second antibody, selected from an antibody specific for a proteasome protein, either for detecting AF-exposing proteasomes, such as AF1, or for detecting intact proteasomes, and an antibody specific for complement factor C3, such as C3, C3c, C3b, iC3b, or, and an antibody specific for complement factor C4, such as C4, C4b, iC4b or C4c. The first and the second antibody are not specific to the same antigen, i.e. that when the first antibody is selected from an antibody specific for a proteasome protein, such as AF1 or intact proteasome, then the second antibody has to be selected from an antibody specific for complement factor C3, such as C3, C3c, C3b, iC3b, or an antibody specific for complement factor C4, such as C4, C4b, iC4b or C4c, or vice versa. If there are more than two different antibodies selected, at least two of those selected need to be selected from antibodies not specific to the same antigen i.e. that when one antibody is selected from an antibody specific for a proteasome protein, such as AF1 or intact proteasome, then at least one other antibody has to be selected from an antibody specific for complement factor C3, such as C3, C3c,C3b, iC3b, or an antibody specific for complement factor C4, such as C4, C4b, iC4b or C4c..

The antibodies can be selected from among commercially available antibodies to a proteasome protein, such as AF1 or intact proteasome, or complement factor C3, such as C3, C3c,C3b, iC3b, or an antibody specific for complement factor C4, such as C4, C4b, iC4b or C4c, and/or an antibody specific for proteasome protein AF1 (such as AF monoclonal antibody (mAb), 3H8 derived from the hybridoma cell culture 2341 (=3H8B3), which is deposited at the DSMZ under the deposition number DSM ACC3271). Such a combination of a first antibody and a second antibody enables a rapid measurement of proteasome-C3 and/or proteasome-C4 complex-formation in bodily fluids, as well as a rapid measurement of AF-exposing proteasomes in bodily fluids. E.g. a first antibody is preferably selected from the group consisting of an antibody specific for proteasome protein AF1 or any other proteasome epitope and the second antibody is preferably selected from the group consisting of an antibody specific for complement factor C3, such as C3, C3c, C3b, iC3b, or an antibody specific for complement factor C4, such as C4, C4b, iC4b or C4c.

In particular, the measurement time of AF in bodily fluids is shortened to several tens of minutes, such as to 1-30, 5-15, 10-20, 10-30, 10-60 minutes, or no more than 10, 20, 30, 40, 50, 60, 90, 120 or 180 minutes, while the measurement of AF in bodily fluids has conventionally required nearly one day. What is more, the present immunological assay and/or the present immunological assay kit enable the skilled practitioner to measure the level of AF in a much smaller aliquot of fluid sample, than was prior needed.

It is further possible to measure the general reaction between proteasomes and complement C3 or C4 with antibodies directed on one hand against proteasome epitopes and on the other hand antibodies directed against epitopes on complement factor 3 or 4.

Consequently, in three specific embodiments, the present invention relates to a kit for detecting 1) AF1-C3 interaction, 2) intact proteasome-C3 complex-formation and/or 3) intact proteasome-C4 complex-formation in bodily fluids, comprising 1) an antibody specific for an AF-exposing proteasome protein, such as AF, and an antibody specific for complement factor C3, or derivates of C3, 2) an antibody specific for a proteasome protein of intact proteasomes and an antibody specific for complement C3, or derivates of C3, and 3) an antibody specific for a proteasome protein of intact proteasomes and an antibody specific for complement C4, or derivates of complement C4. a.

In one embodiment, the kit is an immunological assay kit for determining the presence or absence, and/or the concentration of proteasome-C3-complex-formation or of proteasome-C4-complex-formation in bodily fluids using a first antibody and a second antibody, wherein the first antibody is immobilized on a carrier and the second antibody is modified with a labeling substance, and the first antibody and the second antibody are selected from an antibody specific for a proteasome protein, such as AF1 or intact proteasome, and an antibody specific for complement factor C3,or derivates of C3 or complement factor C4, or derivates of C4.

Derivates of complement factor C3 or C4 are in the present context selected from the group consisting of C3c,C3b, iC3b, C4b, iC4b and C4c.

An immunological assay kit according to the present invention can be an ELISA test kit.

In one embodiment of the present invention, an immunological assay kit according to the present invention comprises an antibody specific for proteasome protein AF1, which is a monoclonal antibody 3H8, herein referred to as mAb 3H8 (AF monoclonal antibody (mAb), 3H8 derived from the hybridoma cell culture 2341 (=3H8B3), which is deposited at the DSMZ under the deposition number DSM ACC3271). In one embodiment of the present invention, an immunological assay kit according to the present invention comprises an antibody specific for complement factor C3c, which is a polyclonal antibody specific for complement factor C3c.

One embodiment of the present invention relates to an immunological assay kit according to the present invention comprising an antibody specific for proteasome protein AF1 (e.g. AF monoclonal antibody (mAb), 3H8 derived from the hybridoma cell culture 2341 (=3H8B3), which is deposited at the DSMZ under the deposition number DSM ACC3271) and an antibody specific for complement factor C3c, which is a polyclonal antibody specific for complement factor C3c.

Further envisioned herein is an immunological assay for determining the presence or absence, and/or the concentration, of proteasome-C3 complex-formation or proteasome-C4 complex-formation in bodily fluids using a first antibody and a second antibody, wherein the first antibody is immobilized on a carrier and the second antibody is modified with a labeling substance, and the first antibody and the second antibody are selected from an antibody specific for a proteasome protein, such as AF1 (e.g. (AF monoclonal antibody (mAb), 3H8 derived from the hybridoma cell culture 2341 (=3H8B3), which is deposited at the DSMZ under the deposition number DSM ACC3271) or intact proteasome and an antibody specific for complement factor C3, such as C3, C3c,C3b, iC3b, or an antibody specific for complement factor C4, such as C4, C4b, iC4b or C4c. Such an immunological assay can be an ELISA test. A first antibody is preferably selected from the group consisting of an antibody specific for proteasome protein AF1 (such as, an antibody specific for LMP2, an antibody specific for 20Salfa6, an antibody specific for 20Salfa1, 2, 3 or 4 and an antibody specific for Rpt5, and the second antibody is preferably selected from the group consisting of an antibody specific for complement factor C3, such as C3, C3c,C3b, iC3b, or an antibody specific for complement factor C4, such as C4, C4b, iC4b or C4c.

In one embodiment of the present invention, an immunological assay according to the present invention comprises an antibody specific for proteasome protein AF1. I a presently preferred embodiment, the antibody selected is AF monoclonal antibody (mAb), 3H8 derived from the hybridoma cell culture 2341 (=3H8B3), which is deposited at the DSMZ under the deposition number DSM ACC3271.

In one embodiment of the present invention, an immunological assay according to the present invention comprises an antibody specific for complement factor C3 which is a polyclonal antibody specific for complement factor C3.

In one embodiment of the present invention, an immunological assay according to the present invention comprises an antibody specific for complement factor C3c which is a polyclonal antibody specific for complement factor C3c.

In one embodiment of the present invention, an immunological assay according to the present invention comprises an antibody specific for complement factor C4 which is a polyclonal antibody specific for complement factor C4.

A method for detecting proteasome-C3 complex-formation in bodily fluids is further disclosed, comprising subjecting a bodily fluid to an immunological assay comprising an antibody specific for a proteasome protein, such as AF1 or intact proteasome and an antibody specific for complement factor C3, such as C3c. An antibody is preferably selected from the group consisting of an antibody specific for proteasome protein AF1, an antibody specific for LMP2,an antibody specific for 20Salfa6, an antibody specific for 20Salfa1, 2, 3 or 4 and an antibody specific for Rpt5, and the other antibody is preferably selected from the group consisting of an antibody specific for complement factor C3, such as C3, C3c,C3b, iC3b.

A method for detecting proteasome-C4 complex-formation in bodily fluids is further disclosed, comprising subjecting a bodily fluid to an immunological assay comprising an antibody specific for a proteasome protein, such as AF1 or intact proteasome and an antibody specific for complement factor C4, such as C4c. An antibody is preferably selected from the group consisting of an antibody specific for proteasome protein AF1, an antibody specific for LMP2, an antibody specific for 20Salfa6, an antibody specific for 20Salfa1, 2, 3 or 4 and an antibody specific for Rpt5, and the other antibody is preferably selected from the group consisting of an antibody specific for complement factor C4, such as C4, C4b, iC4b or C4c.

A method for detecting proteasome-C3 complex-formation and/or proteasome-C4 complex-formation in bodily fluids according to the present invention can be performed as an ELISA test.

In one embodiment of the present invention, said method for detecting proteasome-C3 complex-formation in bodily fluids comprises the use of an antibody specific for proteasome protein AF1 (e.g. AF monoclonal antibody (mAb), 3H8 derived from the hybridoma cell culture 2341 (=3H8B3), which is deposited at the DSMZ under the deposition number DSM ACC3271).

In one embodiment of the present invention, said method for detecting proteasome-C3 complex-formation in bodily fluids comprises the use of an antibody specific for complement factor C3 which is a polyclonal antibody specific for complement factor C3.

In one embodiment of the present invention, said method for detecting proteasome-C3 complex-formation in bodily fluids comprises the use of an antibody specific for complement factor C3c which is a polyclonal antibody specific for complement factor C3c.

In one embodiment of the present invention, a method for detecting proteasome-C4 complex-formation in bodily fluids comprises the use of an antibody specific for complement factor C4 which is a polyclonal antibody specific for complement factor C4.

A kit, an assay and/or a method disclosed herein can be employed to measure, determine, monitor and/or detect proteasome-C3 complex-formation and/or proteasome-C4 complex formation in a vast variety of bodily fluids selected from the group consisting of blood, plasma, urine, milk, saliva, egg yolk, tear fluid, seminal fluid, vaginal fluid, sputum, synovial fluid, gastric fluid, cerebrospinal fluid, eye fluid, pus and mucus.

The present invention further comprises the use of a kit, an assay and/or a method as described herein for detecting, measuring, determining and/or monitoring levels of inflammation in the body of a mammalian, as well as for detecting, measuring, determining and/or monitoring complement system regulation, such as down-regulation, in the body of a mammalian.

What is more, the present invention in one embodiment comprises the use of a kit, an assay and/or a method according to the present invention for verifying effectiveness of processed cereals (SPC) and/or functional food with high levels of natural antisecretory protein (NASP), such as egg-yolk, and/or compliance of human and/or animals to processed cereals (SPC) and/or functional food with high levels of natural antisecretory protein (NASP), such as egg-yolk.

Hereinafter, the embodiments of the present invention will be described in detail. It is to be noted that the embodiments individually disclosed below are examples of the immunological assay and the immunological assay kit and the method and intended use of the present invention. The present invention is not limited to these examples.

### FIGURE LEGENDS

FIG. 1 Detection of antisecretory factor (AF) in human blood plasma by two different methods. Test persons 4-6 had consumed specially processed cereals (SPC), persons 1-3 were controls. In FIG. 1a, the plasma was purified on agarose gel before immune detection with monoclonal antibodies against protein subunit AF1 (PSMD4). In FIG. 1b, plasma was developed directly in a double ELISA, detecting proteasome-complement C3 complexes as described in table 1. Catching antibody was either anti-AF1 (bright column) or anti-LMP2 (black column); detecting antibody was anti-C3c. The Y-axes value is given as reversed titer.

FIG. 2 Western blot of blood plasma from healthy persons using antibodies against C3c. Persons 1 and 2 are controls; person 3 and 4 consumed specially processed cereals (SPC). In the test persons, a partial conversion of C3 into larger and smaller peptides was observed. Well 5 shows plasma from person 1 after incubation in vitro with agarose gel for 4 hours at 37° C., revealing a total conversion of α- into c-peptide. The molecular weights of the α-, β- and c-peptides are 115, 75 and 43 kDa, respectively.

FIG. 3 Western blot of proteasome-attached complement 3 protein. Blood plasma was incubated with agarose gel to obtain aggregated proteasomes and C3 proteins. The proteasomes were subsequently purified by a matrix-bound anti-proteasome antibody. The figure shows α and β subunits of C3 which were co-purified with the proteasomes (row 2; reference in row 1).

FIG. 4 Table 1

Effect of stimulation by agarose on AF1/C3 aggregation as determined in sandwich ELISA.

Persons 1-3 are controls; persons 4-6 consumed processed cereals. Incubation with agarose gel substantially stimulated the proteasome binding to C3 in all samples. Data given as reversed titer.

FIG. 5 Table 2

Concentration of complement factor I (CFI) and H (CFH) in blood plasma.

Persons 1-3 are controls; persons 4-6 had consumed processed cereals. There is no significant difference in factor I or factor H concentration in the test group compared to the control group. Data given as reversed titer.

FIG. 6 SD rats challenged 4 days intranasally with HSV1 (HSV, N=6) or PBS as control (CTR, N=5). Double ELISA with monoclonal antibodies against proteasome subunits (205α4, 20Sα6, LMP2, LMP7, RPT5) as catching antibody and polyclonal antibodies against complement C3 as detecting antibody. In plasma and liquor the complex of 20Sα6 subunits and C3 was significantly higher (p<0,05 and p<0,01,resp) the LMP2 or 20Sα4 and complement C3 significantly higher in plasma.

FIG. 7 SD rats challenged 4 days intranasally with HSV1 (HSV) or PBS as control (CTR). Double ELISA with monoclonal antibodies against proteasome subunits (205α6) as catching antibody and polyclonal antibodies against complement C4 as detecting antibody. In plasma and liquor the complex of 20Sα6 subunits and C4 was significantly higher (p<0,05).

FIG. 8 2D analyse of agarose-purified plasma. Samples were taken from three individuals, 1-3, before (A) and after 4 weeks of SPC-intake (B). Immoboline drystrips pH 3-10 was used for isoelectric focusing and 10% tris-glycine gel for second dimension. To the left is the molecular weight standard applied and the 40 kDa band is shown. The protein content was visualized by silver staining. Selected spot for subsequent analyse is indicated with arrow.

FIG. 9 Table 3

Summary of LC-MS/MS identification for picked spot in silver stained gel after 2D separation of agarose-purified plasma after SPC induction of AF. Contamination proteins have been removed from the results. *=Sequence modifications with propionamide are inserted as letter c.

FIG. 10 Western blotting on 2D gel with separated agarose-purified plasma after SPC induction of AF. The first dimension electrophoresis isoelectric focusing was run on 7 cm strip gel, pH 3-10, while 10% tris-glycine gel was used for the second dimension. The PVDF membrane was incubated with anti-C3c. To the left is the molecular weight standard applied showing the 40 and 80 kDa bands.

FIG. 11 The level of C3c in agarose-purified plasma (n=4) tested by ELISA using anti-C3c. Samples from before and after 6 weeks of SPC-intake were analyzed and net absorbance level were determined. Data are shown as mean±SEM. **Significant difference (p=0.0039) between samples taken before and after the SPC diet.

FIG. 12 ELISA determination of AF-activity and of complement factors. The level of AF-activity and complement factors determined with the (A) monoclonal antibody AF 3H8 (AF monoclonal antibody (mAb), 3H8 derived from the hybridoma cell culture 2341 (=3H8B3), which is deposited at the DSMZ under the deposition number DSM ACC3271) and antibodies reacting against C3c, C4c or Factor H (B, C, D) in agarose-purified plasma from three individuals before, during and one week after intake of SPC. Data are expressed as mean±SEM. Significant differences between the samples are visualized by *=p<0.05 and **=p<0.01.

FIG. 13 Western blot analysis using anti-C3c detection in human plasma. Lane 1: direct plasma before SPC-intake. Lane 2: direct plasma after SPC-intake. Lane 3: agarose-purified plasma before SPC-intake. Lane 4: agarose-purified plasma after SPC-intake. In the centre is the molecular weight standard applied, the 43 and 75 kDa bands are marked.

### DEFINITIONS AND ABBREVIATIONS

Abbreviations

IFP: interstitial fluid pressure;

PBS: phosphate buffered saline;

AF: antisecretory factor, Full-length AF protein (as shown in SEQ ID NO: 1)

AF-6: a hexa peptide CHSKTR (as shown in SEQ ID NO: 2);

AF-16: a peptide composed of the amino acids VCHSKTRSNPENNVGL (as shown in SEQ ID NO: 3);

AF-8: a septa peptide VCHSKTR (as shown in SEQ ID NO: 4);

Octa peptide IVCHSKTR (as shown in SEQ ID NO: 5);

Penta peptide HSKTR (as shown in SEQ ID NO: 6);

SPC: Specially Processed Cereals;

RTT: Method for measuring a standardized secretion response in rat small intestine, as published in SE 9000028-2 (publication number 466331) for measuring content of AF (ASP);

AF: Antisecretory Factor;

ELISA: Enzyme-linked immunosorbent assay;

PBS: phosphate buffered saline;

AP: alkaline phosphatase;

BSA: bovine serum albumin;

mAb: monoclonal antibody;

LC-MS/MS: nanoflow liquid chromatography-tandem mass spectrometry;

PAGE: polyacrylamide gel electrophoresis.

HSV1: herpes simplex virus-1

Definitions

Proteins are biological macromolecules constituted by amino acid residues linked together by peptide bonds. Proteins, as linear polymers of amino acids, are also called polypeptides. Typically, proteins have 50-800 amino acid residues and hence have molecular weights in the range of from about 6,000 to about several hundred thousand Dalton or more. Small proteins are called peptides, polypeptides, or oligopeptides. The terms “protein”, “polypeptide”, “oligopeptide” and “peptide” may be used interchangeably in the present context. Peptides can have very few amino acid residues, such as between 2-50 amino acid residues (aa).

The term “antisecretory” refers in the present context to inhibiting or decreasing secretion and/or fluid transfer. In the present context, the terms an “Antisecretory factor protein”, “antisecretory factor (AF) protein”, “AF-protein”, AF, or a homologue, derivative or fragment thereof, may be used interchangeably with the term “antisecretory factors” or “antisecretory factor proteins” as defined in WO 97/08202, and refer to an antisecretory factor (AF) protein or a peptide or a homologue, derivative and/or fragment thereof having antisecretory and/or equivalent functional and/or analogue activity, or to a modification thereof not altering the function of the polypeptide. Hence, it is to be understood that an “antisecretory factor”, “antisecretory factor protein”, “antisecretory peptide”, “antisecretory fragment”, or an “antisecretory factor (AF) protein” in the present context, also can refer to a derivative, homologue or fragment thereof. These terms may all be used interchangeably in the context of the present invention. Furthermore, in the present context, the term “antisecretory factor” may be abbreviated “AF”. Antisecretory factor (AF) protein in the present context also refers to a protein with antisecretory properties as previously defined in WO97/08202 and WO 00/38535. Antisecretory factors have also been disclosed e.g. in WO 05/030246.

SPC© is a medical food comprising specially processed cereals (SPC).

A “medical food”, in the present context, refers to a food, a feed or food supplement, or a food for special dietary use, which has been prepared with an antisecretory factor (AF) protein, or alternatively, has the capability to induce synthesis and/or activation of endogenous AF. Said food may be any suitable food, in fluid or solid form, such as a liquid or a powder, or any other suitable foodstuff. Examples of such matter may be found in WO 0038535 or WO 91/09536.

Salovum© Also intended by the term antisecretory factor are native antisecretory factors (NASP) which can be provided in egg yolk with a high content of antisecretory factors (NASP), as e.g. disclosed in SE 900028-2 and WO 00/38535, and as further described below.

Compliance is in the present context employed to describe the degree of constancy and accuracy with which a patient follows a prescribed regimen, as distinguished from adherence or maintenance It encompasses the patients active participation in his or her own healthcare; seeking medical advice, keeping appointments, following recommendations concerning lifestyle, as well as following medical regimens.

### DETAILED DESCRIPTION OF THE INVENTION

Clinical studies have shown that patients with chronic diseases, involving inflammation and/or secretory disturbances, have a low plasma AF-activity (Lange et al., 2003; Laurenius et al.,2003). Thus, in chronic affections, the disease process does not appear to be a sufficient stimulus for an endogenous increase in AF-activity. Still, these patients may benefit from a diet-induced raise in AF-activity.

Previous studies by the present inventors, using polyclonal antibodies, indicate that there exist a number of conformational variants of AF in tissues (Jennische et al., 2006), and it was suggested that regulation of the AF-activity involves conformational changes, which exposes or hides the active sequence(s) of the AF protein. Most of the AF present in plasma is in an antisecretory inactive state, and activation of a fraction of the AF occurs as a response to intestinal challenge with bacterial toxins, or to specific dietary compounds. The present disclosure proves that activation steps involve exposure of the antisecretory site of the AF1 protein in the proteasome.

The present inventors have previously shown that increased binding of AF1 to agarose is connected to its antisecretory and anti-inflammatory activity. Surprisingly it is herein disclosed that consumption of SPC not only leads to binding of AF1 to agarose, but also to complement factor C3. The association between AF1 and C3 can be demonstrated by a sensitive ELISA test requiring only minimal amounts of blood plasma. The formerly used test requires 10 times more plasma and is more time consuming, since a primary purification on agarose columns has to be performed. The results further show that not only the regulatory 19S subunit, but also the catalytic 20S subunit, associates with C3.

The herein for the first time disclosed monoclonal AF1 antibodies (AF monoclonal antibody (mAb), 3H8 derived from the hybridoma cell culture 2341 (=3H8B3), which is deposited at the DSMZ under the deposition number DSM ACC3271) did not react with intact proteasomes, indicating that an opening of the protein structure occurs at the binding of C3 after intake of SPC. Thus indicating that normally, AF1 is hidden between the 19S and 20S proteasome subunits, but the reaction with C3c seems to open up the peptide sequence responsible for the antisecretory activity of the AF complex.

The binding to proteasomes can lead to a conversion of C3 into its inactive form, C3c. The split of C3 into C3c has previously been shown to always involve the proteolytic complement factor I, with factor H as a cofactor. Whether circulating proteasomes also act as cofactors, or rather are directly involved in the proteolytic splitting, remains to be seen. The ability of agarose to convert the entire a-chain of C3 into C3c is remarkable. Prior to the present disclosure, only a partial conversion into C3c has been described as blood plasma was exposed to different surface active materials.

What is more, for the first time it is shown that virus infection induces complex formation between proteasomes and complement factors C3 and/or C4. Intracellular proteasomes play an important role during initiation of inflammation, e.g. during activation of NFκB. Inflammasome priming by lipopolysaccharide is dependent upon proteasome function. Without wishing to limit the present invention to a scientific hypothesis, possibly, proteasomes in the blood might trigger inflammation by enhancing the complement action. On the other hand SPC counteracts this by causing splitting of proteasomes as well as complement which causes down-regulation of proteasomes and C3 complement factor. This hypothesis is strengthened by recent experiments with proteasome inhibitory food components, which act anti-inflammatory in patients. Furthermore, certain food components have the ability to decrease C3 levels in blood, which might be due to proteasome-C3 aggregation.

There are no previous studies of intact proteasomes in body fluids. However, according to the GeneCards, discrete proteasome proteins like AF1 are assumed to occur in blood plasma in the same concentrations as in tissues, e.g. 1 ppm (http://www.genecards.org/cgi-bin/carddisp.pl?gene=PSMD4). This concentration is higher than the presently presented estimate, as well as the reported level of circulating 20S proteasome subunits. The discrepancy might suggest that proteasomes in blood differ from those in tissues, which are used as standards in the disclosed assays. Interestingly, the concentration of circulating 20S proteasomes has been correlated to autoimmune diseases, such as rheumatoid arthritis, systemic lupus erythematosus and Sjögrens disease. Whether these 20S subunits also may be associated with 19S regulatory subunits is an open question.

In conclusion, for the first time, the existence of a complex of proteasomes and complement factor 3 and a complex of proteasomes and complement factor C4, respectively, is disclosed. This formation is herein induced by herpes simplex virus 1 but might of course be induced by a other inflammatory agents. This reaction is counteracted by consumption of SPC which leads to degradation of the proteasome-complement complex, in which both components are partially split: leading to the proteasome exposing hidden antisecretory peptide sequences and C3 being converted into C3c.

This insight leads to the development of new immunological assays for measuring proteasome-C3 complex-formation contained in a sample utilizing an antigen-antibody reaction, wherein the aforementioned combination of the first antibody and the second antibody is such a combination that enables a rapid measurement of proteasome-C3 complex-formation and/or proteasome-C4 complex-formation in body fluids. In particular, several new ELISA methods are described that can determine 1) proteasome-C3 formation, 2) proteasome C4 formation and 3) AF-exposing proteasome in bodily fluids, as well ascirculating 26S proteasomes in blood plasma.

Immunological Assay

In one embodiment of the present invention, an immunological assay of the present invention is an immunological assay for measuring proteasome-C3 complex-formation contained in a sample utilizing an antigen-antibody reaction, wherein the aforementioned combination of the first antibody and the second antibody is such a combination that enables a rapid measurement of proteasome-C3 complex-formation.

In another embodiment of the present invention, an immunological assay of the present invention is an immunological assay for measuring proteasome-C4 complex-formation contained in a sample utilizing an antigen-antibody reaction, wherein the aforementioned combination of the first antibody and the second antibody is such a combination that enables a rapid measurement of proteasome-C4 complex-formation.

In yet another embodiment of the present invention, an immunological assay of the present invention is an immunological assay for measuring AF1-exposed proteasome-C3 complex-formation contained in a sample utilizing an antigen-antibody reaction, wherein the aforementioned combination of the first antibody and the second antibody is such a combination that enables a rapid measurement of AF1-exposed proteasome-C3 complex-formation.

That is, an immunological assay of the present invention is an immunological assay for measuring proteasome-complement complex-formation, using any one of the possible combinations as a combination of the first antibody binding to a proteasome protein, such as AF1 or intact proteasome, which is a substance to be measured, and the second antibody binding to complement factor C3, or C4, or derivates of C3 or C4. According to the above, an immunological assay of the present invention enables a rapid measurement of proteasome-complement complex-formation contained in a sample.

Examples of an immunological assay include an Enzyme-Linked Immunosorbent

Assay (ELISA, EIA), a fluorescent immunoassay (FIA), a radioimmunoassay (RIA), a luminescence immunoassay (LIA), an enzyme antibody technique, a fluorescence antibody assay, an immunochromatography, an immunonephelometry, a latex turbidimetry, and a latex agglutination assay.

Further, the measurement in an immunological assay of the present invention can be performed manually or using an apparatus such as an analytical apparatus.

Further, an immunological assay of the present invention can be operated according to a publicly known method. For example, the first antibody immobilized on a carrier, a sample, and the second antibody modified with a labeling substance are reacted simultaneously or in sequence. A complex of “the first antibody immobilized on a carrier-and-the second antibody modified with a labeling substance” is formed by the above reaction, and the amount (concentration) of proteasome-complement complex-formation contained in the sample can be measured based on the amount of the second antibody modified with a labeling substance contained in the complex.

For example, an enzyme-linked immunosorbent assay may be carried out using a microplate on which the first antibody is immobilized, a diluted solution of specimen, the second antibody modified with an enzyme such as HRP, a wash buffer, and a substrate solution. Further, the measurement may be performed by allowing the enzyme modifying the second antibody to react with its substrate under the optimum conditions for the enzyme and measuring the amount of the product of the enzyme reaction by an optical method and the like.

Also, a fluorescent immunoassay may be performed using an optical waveguide on which the first antibody is immobilized, a diluted solution of specimen, the second antibody modified with a fluorescent substance, and a wash buffer. Also, the measurement may be performed by irradiating the fluorescent substance modifying the second antibody with the excitation light, and measuring the intensity of the fluorescence emitted by the fluorescent substance.

Further, when a radioimmunoassay is carried out, the amount of radiation emitted by a radioactive substance is measured. Also, when a luminescence immunoassay is carried out, the amount of light emitted from a luminescent reaction system is measured.

Further, when an immunonephelometry, a latex turbidimetry, a latex agglutination assay, and the like are carried out, the transmitted light and the scattering light are measured by an endpoint method or a rate method. Also, when an immunochromatography and the like are visually carried out, the color of a labeling substance appearing on the test line is visually measured. It is to be noted that an instrument such as an analytical apparatus can be used instead of the visual measurement.

A sample to be used in the immunological assay or method of the present invention includes all of the biological samples possibly containing a proteasome-complement complex such as a body fluid including blood, serum, plasma, milk, tear fluid, tear, semen, seminal fluid, vaginal fluid, saliva, sputum, sweat, ascites, amniotic fluid synovial fluid, gastric fluid, cerebrospinal fluid, spinal fluid, eye fluid, pus and/or mucus of a mammal.

A sample to be used in the immunological assay or method of the present invention can be harvested from any mammal. In one embodiment, the mammal is selected from the group consisting of a human, a horse, a cow, a pig, a sheep, a goat, a rodent, a dog, a cat or a camel.

In a specific embodiment of the present invention, a sample to be used in the immunological assay or method of the present invention can be harvested from a bird, such as a hen. The person skilled in the art will know that in this specific embodiment, antibodies against bird antigens need to be selected. Thus, the antibodies selected are a first antibody binding to a bird proteasome protein, such as AF1 or intact proteasome, which is a substance to be measured, and the second antibody binding to bird complement factor C3 or a derivate thereof, or to complement factor C4 or a derivative thereof. A sample to be used in this specific embodiment of immunological assay or method of the present invention includes all of the biological samples possibly containing proteasome-complement complex such as a body fluid including in particular blood, serum, plasma, and egg yolk of a bird, such as but not limited to a hen.

Immunological Assay Kit

An immunological assay kit of the present invention is an immunological assay kit for measuring proteasome-C3 complex-formation, proteasome-C4 complex-formation and AF1-exposed proteasome-C3c complex-formation contained in a sample utilizing an antigen-antibody reaction, wherein the aforementioned combination of the first antibody and the second antibody is such a combination that enables a rapid measurement of proteasome-complement complex-formation.

An immunological assay kit according to the present invention can be used for the aforementioned immunological assay of the present invention. Accordingly, a similar measurement principle and the like to the aforementioned immunological assay apply to an immunological assay kit of the present invention.

Other Reagent Components in the Immunological Assay Kit

In an immunological assay kit of the present invention, various aqueous solvents can be used as a solvent. Examples of the aqueous solvent can include purified water, physiological saline, or various buffers such as a tris buffer, a phosphate buffer, or a phosphate-buffered physiological saline. No particular limitation is imposed on a pH of these buffers, and a suitable pH may be appropriately selected; however, the pH is generally selected within a range of pH 3 to 12.

Further, an immunological assay kit of the present invention may appropriately include, in addition to the aforementioned first antibody immobilized on a carrier and the second antibody modified with a labeling substance, one or more kinds of a protein such as bovine serum albumin (BSA), human serum albumin (HSA), casein, or a salt thereof, various salts, various sugars, powdered skim milk, sera of various animals such as normal rabbit serum, various preservatives such as sodium azide and an antibiotic, an activator, a reaction promoter, a sensitivity enhancer such as polyethylene glycol, a non-specific reaction inhibitor, various surfactants such as a non-ionic surfactant, an ampholytic surfactant, or an anionic surfactant, and the like. Although no particular limitation is imposed on the concentrations of these substances in an assay reagent, the concentrations can be 0.001 to 10% (W/V), can particularly be 0.01 to 5% (WN).

Composition of the Immunological Assay Kit

No particular limitation is imposed on an immunological assay kit of the present invention as long as the aforementioned first and second antibodies include at least one of first antibody binding to a proteasome protein, such as AF1 or intact proteasome, which is a substance to be measured, and the second antibody binding to complement factor C3, such as C3, C3c,C3b, iC3b, or an antibody specific for complement factor C4, such as C4, C4b, iC4b or C4c. Typically, the at least first and second antibodies are provided in separate containers.

Further, an immunological assay kit of the present invention for sale may include, in addition to a reagent containing the aforementioned two antibodies, other reagents in combination and separated.

Examples of the aforementioned other reagents include a buffer, a diluted solution of sample, a diluted solution of reagent, a reagent containing a labeling substance, a reagent containing a substance generating a signal such as color development, a reagent containing a substance involved in generation of a signal such as color development, a reagent containing a substance for calibration, a reagent containing a substance used for accuracy control.

Then, the aforementioned other reagents and the immunological assay reagent of the present invention may be appropriately used and sold in various combinations, for example, the other reagents may be provided as a first reagent and the immunological assay reagent of the present invention may be provided as a second reagent, or the immunological assay reagent of the present invention may be provided as a first reagent and the aforementioned other reagents may be provided as a second reagent.

Also, while no particular limitation is imposed on the configuration of an immunological assay kit of the present invention, in order to perform a rapid measurement in a simple manner, an immunological assay kit of the present invention can be provided as an all-in-one diagnostic kit, in which the components of the immunological assay kit of the present invention are integrated. Although no particular limitation is imposed on the aforementioned all-in-one diagnostic kit, examples thereof include an ELISA kit, a fluorescent immunoassay kit, and an immunochromatography kit.

For example, a configuration of an ELISA kit includes a microplate on which the first antibody is immobilized, a diluted solution of specimen, the second antibody modified with an enzyme such as HRP, a wash buffer, a substrate solution, and the like.

Further, when a fluorescent immunoassay kit is provided, the kit includes an optical waveguide on which the first antibody is immobilized, a diluted solution of specimen, the second antibody modified with a fluorescent substance, a wash buffer, and the like.

Further, when an immunochromatography kit is provided, the following embodiment may be provided as an example. A membrane having the aforementioned first antibody immobilized on one end (downstream side) thereof is stored in a reaction cassette. Meanwhile, a developing solution is set on the other end (upstream side) of the membrane, and a pad having a substrate for the aforementioned labeling substance added thereto is arranged in the downstream side near where the developing solution is set, and a pad having the second antibody labeled with the aforementioned labeling substance is arranged in the intermediate part of the membrane.

Antibody

The first antibody in the immunological assay and the immunological assay kit of the present invention is immobilized on a carrier. That is, the first antibody is prepared by allowing one of the antibodies to adsorb or bind to a carrier through physisorption, chemical binding, or a method such as a combination of these.

The antibody immobilized by physisorption can be prepared according to a publicly known method. Examples of such a method include a method in which the antibody and a carrier are mixed and contacted with each other in a solution such as a buffer and a method in which the antibody dissolved in a buffer and the like is allowed to contact a carrier.

Typically, the first antibody and the second antibody are selected from an antibody specific for a proteasome protein, such as AF1 (e.g. (AF monoclonal antibody (mAb), 3H8 derived from the hybridoma cell culture 2341 (=3H8B3), which is deposited at the DSMZ under the deposition number DSM ACC3271) or intact proteasome and an antibody specific for complement factor C3, such as C3, C3c,C3b, iC3b, or an antibody specific for complement factor C4, such as C4, C4b, iC4b or C4c.

A first antibody is preferably selected from the group consisting of an antibody specific for proteasome protein AF1 (such as, AF monoclonal antibody (mAb), 3H8 derived from the hybridoma cell culture 2341 (=3H8B3), which is deposited at the DSMZ under the deposition number DSM ACC3271), an antibody specific for LMP2, an antibody specific for 20Salfa6, an antibody specific for 20Salfa1, 2, 3 or 4 and an antibody specific for Rpt5, and the second antibody is preferably selected from the group consisting of an antibody specific for complement factor C3, such as C3, C3c,C3b, iC3b, or an antibody specific for complement factor C4, such as C4, C4b, iC4b or C4c..

Enclosed in the present application is a depositor's statement of authorization and consent testifying that Anders Oldfors, Dept. Pathology, Institute of Biomedicine, University of Göteborg, P.O.B. 420, S-40530 Göteborg, Sweden, has in the name of the Department of infectious Diseases, institute of Biomedicine, Göteborg University, Guidhedsgatan 10, 41346 Göteborg, Sweden, on May 12th, 2015, deposited with Leibniz-lnstitute DSMZ-Deutsche Sammlung von Mikroorganismen and Zellkulturen GmbH, Inhoffenstr. 7 B, D-38124 Braunschweig, Germany, under accession number DSM ACC3271 biological material on the same terms as those laid down in the Budapest Treaty. The depositor has herein authorized Lantmännen AS-Faktor AB, BOX 30192, 104 25 Stockholm, to refer to the afore-mentioned deposited biological material in the international and European and U.S. patent applications No.[ representative's reference number PS54728PC00/PS54728EP00/PS54728US00] and given his unreserved and irrevocable consent to the deposited material being made available to the public in accordance with Rule 33 EPC as from the date of filing of the aforementioned patent applications.

Further, the antibody immobilized by chemical binding can also be prepared according to a publicly known method. Examples of such a method include a method in which the antibody and a carrier are mixed with a divalent cross-linking reagent such as glutaraldehyde, carbodiimide, imide ester, or maleimide and contacted with each other so that amino groups, carboxyl groups, thiol groups, aldehyde groups, hydroxyl groups, or the like of both of the antibody and the carrier react.

Further, a treatment for inhibiting a non-specific reaction, spontaneous agglomeration of the carrier on which the antibody is immobilized, and the like can be performed according to a publicly known method, if needed. Examples of such a method include a method in which the surface or the inner surface of the carrier on which the antibody is immobilized is contacted with a protein such as bovine serum albumin (BSA), casein, gelatin, egg albumin, or a salt thereof, a surfactant, powdered skim milk, or the like so that the surface or the inner surface of the carrier is covered with these substances. The second antibody in the immunological assay and the immunological assay kit of the present invention is modified with a labeling substance. The second antibody is prepared by allowing one of the antibodies to adsorb or bind to a labeling substance through physisorption, chemical binding, or a method such as a combination of these. The antibody having a labeling substance bound thereto by physisorption can be prepared according to a publicly known method. Examples of such a method include a method in which the antibody and a labeling substance are mixed and contacted with each other in a solution such as a buffer and a method in which the antibody dissolved in a buffer and the like is allowed to contact a labeling substance.

For example, when the labeling substance is gold colloid or latex, physisorption is effective. An antibody labeled with gold colloid is obtainable by mixing the antibody and gold colloid in a buffer and allowing them to contact each other.

Further, the antibody modified with a labeling substance by chemical binding can also be prepared according to a publicly known method. Examples of such a method include a method in which the antibody and a labeling substance are mixed with a divalent cross-linking reagent such as glutaraldehyde, carbodiimide, imide ester, or maleimide and contacted with each other so that amino groups, carboxyl groups, thiol groups, aldehyde groups, hydroxyl groups, or the like of both of the antibody and the labeling substance react. For example, when the labeling substance is a fluorescent substance, an enzyme, or a chemiluminescent substance, chemical binding is effective.

Further, a treatment for inhibiting a non-specific reaction, spontaneous agglomeration of the antibody modified with labeling substances, and the like can be performed according to a publicly known method, if needed. Examples of such a method include a method in which the antibody having a labeling substance bound thereto is contacted with a protein such as bovine serum albumin (BSA), casein, gelatin, egg albumin, or a salt thereof, a surfactant, powdered skim milk, or the like so that the antibody is covered with these substances.

Also, when an enzyme-linked immunosorbent assay is carried out, peroxidase (POD), alkaline phosphatase (ALP), β-galactosidase, urease, catalase, glucose oxidase, lactate dehydrogenase, amylase, or the like can be used as a labeling substance.

Also, when a fluorescent immunoassay is carried out, fluorescein isothiocyanate, tetramethylrhodamine isothiocyanate, substituted rhodamine isothiocyanate, dichlorotriazine isothiocyanate, cyanine, merocyanine, or the like can be used. Also, when a radioimmunoassay is carried out, tritium, iodine-125, iodine-131, or the like can be used.

Also, when a luminescence immunoassay is carried out, a luminol compound, a luciferase compound, an acridinium ester, a dioxetane compound, or the like can be used.

Also, when an immunochromatography, an immunonephelometry, a latex turbidimetry, and a latex agglutination assay are carried out, particles made of a material such as polystyrene, a styrene-styrene sulfonate copolymer, an acrylonitrile-butadiene-styrene copolymer, a vinyl chloride-acrylic acid ester copolymer, a vinyl acetate-acrylic acid copolymer, polyacrolein, a styrene-methacrylic acid copolymer, a styrene-glycidyl (meth)acrylic acid copolymer, a styrene-butadiene copolymer, a (meth)acrylic acid polymer, an acrylic acid polymer, latex, gelatin, a liposome, a microcapsule, silica, alumina, carbon black, a metal compound, metal, metal colloid, a ceramic, or a magnetic material can be used.

As the carrier in the immunological assay of the present invention, a solid carrier in the form of a bead made of a material such as polystyrene, polycarbonate, polyvinyl toluene, polypropylene, polyethylene, polyvinyl chloride, nylon, polymethacrylate, polyacrylamide, latex, a liposome, gelatin, agarose, cellulose, sepharose, glass, metal, a ceramic, or a magnetic material, a microplate, a test tube, a stick, a membrane, a specimen piece, or the like can be used. Specifically, as the carrier of the present invention, a waveguide configured as an optical waveguide can be used.

The Antisecretory Factor

The antisecretory factor is a class of proteins that occurs naturally in the body. The human antisecretory factor AF protein is a 41 kDa protein, comprising 382-288 amino acids when isolated from the pituitary gland. The active site can be localized to the protein in a region close to the N-terminal of the protein, in particular localized to amino acids 1-163 of SEQ ID NO 1, more specifically to amino acid positions 35-50 on the antisecretory factor (AF) protein sequence. The biological effect of AF is exerted by fragments and/or homologues comprising at least 6 amino acids, SEQ ID NO: 2 (AF-6), of said consensus sequence, or a modification thereof not altering the function of the polypeptide and/or peptide.

The present inventors have shown that the antisecretory factor is homologous with the protein S5a, and Rpn10, which constitutes a subunit of a constituent prevailing in all cells, the 26 S proteasome, more specifically in the 19 S/PA 700 cap. In the present invention, antisecretory factor (AF) proteins are defined as a class of homologue proteins having the same functional properties. Antisecretory factor is also highly similar to angiocidin, another protein isoform known to bind to thrombospondin-1 and associated with cancer progression.

A present immunological assay, as well as a present immunological assay kit and method described herein, use at least a first antibody and a second antibody, one of which is selected from an antibody specific for a proteasome protein, such as AF1 or intact proteasome, a homologue to AF, a derivative, a peptide, or a fragment of antisecretory factor (AF), which has analogous biological activity. A homologue, derivative or fragment, in the present context, comprises at least 6 amino acids (as shown in SEQ ID NO: 2) corresponding to those of a naturally occurring antisecretory factor (AF) protein, which may be further modified by changing one or more amino acids in order to optimize the antisecretory factor's biological activity, without altering the essential function of the polypeptide and/or peptide.

WO 00/038535 discloses food products, enriched in antisecretory factor (AF) proteins as such, which are examples for suitable food, foodstuff and/or food supplements in the present context.

The present invention for the first time discloses an immunological assay, method and kit for determining the presence or absence, and/or the concentration, of AF1-exposed proteasome-C3 complex formation in bodily fluids using a first antibody and a second antibody, wherein either the first antibody or the second antibody is selected from an antibody specific for AF1, a homologue to AF, a derivative, a peptide, or a fragment of antisecretory factor (AF), which has analogous biological activity.

The disclosed assay or method can be used for detecting levels of circulating 26S proteasomes in blood plasma or other body fluids, such as for monitoring levels of inflammation in the body of a mammalian, including complement system (down-) regulation in the body of a mammalian as well as for verifying compliance of processed cereals (SPC) and/or functional food with high levels of natural antisecretory protein (NASP).

In one embodiment, the disclosed immunological assay or method is employed to verify compliance of Salovum© (NASP), a food, feed and/or food supplement, with a very high level of native antisecretory factor (AF) protein, which is preferably provided as egg yolk rich in naturally occurring antisecretory factors. It is e.g. envisaged to test a sample harvested from a subject having consumed said food, feed and/or food supplement to measure proteasome-complement complex-formation in said subject after consumption of said food, feed and/or food supplement.

In another, equally preferred embodiment, the disclosed immunological assay or method is employed to verify compliance of processed cereals, such as SPC©, which stimulate endogenous production of an antisecretory factor (AF) protein as shown in SEQ ID NO: 1 (AF), and/or a homologue and/or fragment thereof having equivalent activity and comprising an amino acid sequences as shown in SEQ ID NO: 2 (AF-6), and/or a pharmaceutically active salt thereof, by the subject after intake of a food and/or a food for special dietary use that induces the uptake, formation and/or release of an antisecretory factor (AF) protein. It is e.g. envisaged to test a sample harvested from a subject having consumed said food, feed and/or food supplement to measure proteasome-complement complex-formation in said subject after consumption of said food, feed and/or food supplement.

Experimental Section

In the present study it is shown that the AF complex consists of both proteasomes and complement C3. Proteasomes and proteasome-complement C3 complexes are purified from human blood and analyzed by western blot and mass-spectroscopy. Antibodies specific for proteasome subunits are produced and two new immunoassays (ELISA) are developed. The first ELISA test establishing a 26S proteasome concentration of 0.41±0.03 μg/ml in normal plasma, the second ELISA disclosing the binding of proteasomes to complement factor C3. The latter test measures AF, which is increased about tenfold following intake of processed cereals. No difference in the levels of complement factors H and I were seen. The induction of AF could be mimicked by incubating blood plasma with agarose gel in vitro, making it possible to isolate and characterize the formed protein peptide sequence. C3 is partially split into its inactive form C3c upon proteasome binding, which explains the anti-inflammatory effect during AF complex formation. In conclusion, 26S proteasomes occur in normal blood and interact with the complement C3, forming the AF complex.

### EXAMPLE 1

Materials and Methods

General Reagents

Monoclonal antibodies specific for AF1 were produced and are deposited under the deposition number XY. Monoclonal mouse IgG antibodies specific for proteasome LMP2 (20Sβ1i) and 20Sα6 were obtained from Enzo Life Science Inc.

(www.enzolifesciences.com). Polyclonal antibodies specific for complement factor C3c were purchased from Dako (www.dako.com). Sepharose 6B and Sephacryl S300 were obtained from Amersham (GE healthcare). Pre-swollen DE52 cellulose was from Whatman no 4057050 Schleicher & Schuell (GE healthcare). Specially processed cereals (SPC) were obtained from AS-Faktor AB, Stockholm, Sweden (www.as-faktor.se).

Human Blood Samples

Six healthy test persons were recruited from the Biomedical department at the University of Gothenburg. The ethical committee of the University of Gothenburg approved the study protocol (Dnr. 037-14, Exp. 2014-02-19). Three of the test persons consumed SPC at 1 g/kg body weight once a day for one month, while the remaining three served as controls. From each person, 15-20 ml venous blood was drawn into EDTA vacutainer tubes (gbo.com/preanalytics). After separation by centrifugation, the plasma was mixed with an equal volume of citrate buffer (glucose 0.11 M, tri-sodium-citrate 0.03 M and sodium chloride 0.07 M, pH 6.1). Each sample was subsequently divided in 2 ml aliquots and kept at −20 C until use. No sample was stored frozen for more than four weeks.

AF1 Assay After Agarose Filtration.

As previously described, AF1 was affinity purified from blood plasma on small agarose columns and concentration determined by immunoassay (Johansson, E., I. Lonnroth, I. Jonson, S. Lange, and E. Jennische, Development of monoclonal antibodies for detection of Antisecretory Factor activity in human plasma. J Immunol Methods, 2009. 342(1-2): p. 64-70.). In short, 6 ml of the 1:1 diluted plasma samples (described above) were run through 3 ml Sepharose 6B columns, washed twice with PBS and subsequently eluted with 1 M α-methyl-glycoside. The purified samples were titrated in a 96-well plate, coated over-night, detected by the 3H8 monoclonal mouse antibody specific for AF1 (AF monoclonal antibody (mAb), 3H8 derived from the hybridoma cell culture 2341 (=3H8B3), which is deposited at the DSMZ under the deposition number DSM ACC3271) or PBS as control, and finally developed with a secondary antibody bound to alkaline phosphate (AP). After absorbance reading at 405 nm the result was given as reversed titer. The 3H8 antibody (AF monoclonal antibody (mAb), 3H8 derived from the hybridoma cell culture 2341 (=3H8B3), which is deposited at the DSMZ under the deposition number DSM ACC3271) recognizes the AF sequence responsible for the antisecretory activity (Nicolas, V. and V. Lievin-Le Moal, Antisecretory peptide AF-16 inhibits the Sat toxin-stimulated transcellular and paracellular passages of fluid in cultured human enterocyte-like cells. Infect Immun, 2014. Matson Dzebo, M., A. Reymer, K. Fant, P. Lincoln, B. Norden, and S. Rocha, Enhanced cellular uptake of antisecretory peptide AF-16 through proteoglycan binding. Biochemistry, 2014. 53(41): p. 6566-73.).

Proteasome/C3 ELISA

A sandwich ELISA was performed for the detection of proteasome/C3 complexes in blood plasma. Monoclonal 3H8 antibodies specific for proteasome proteins AF1 (AF monoclonal antibody (mAb), 3H8 derived from the hybridoma cell culture 2341 (=3H8B3), which is deposited at the DSMZ under the deposition number DSM ACC3271) diluted 1:200 or LMP2 diluted 1:2000 were coated overnight on a 96-well titer plate. After blocking with 0.2% BSA at 37° C. for 45 min, plasma samples were titrated in 0.2% BSA, 0.05% Tween 20, PBS and shaken for 1 hour. A polyclonal rabbit antibody at 1:2000 dilution was applied as detecting antibody. After incubation for 30 min, an anti-rabbit-AP secondary antibody was applied, and after additionally 30 min, AP substrate was added. Absorbance was read at 405 nm in a photometer.

Circulating Proteasomes

19S proteasome subunits were prepared from human erythrocytes as described by DeMartino (DeMartino, G. N., Purification of PA700, the 19S regulatory complex of the 26S proteasome. Methods Enzymol, 2005. 398: p. 295-306.). In short, washed erythrocytes from human blood were lysed in 20 mM Tris-HCl, 1mM EDTA, 5 mM mercaptoethanol and centrifuged at 10 000×g for 2 hours. To the lysate, ¼ volume of DE52 cellulose was added and slowly mixed at room temperature for 30 min. The cellulose was filtered away and the filtrate precipitated in 40% saturated ammonium sulfate. After dissolving the precipitate in buffer X (20 mM Tris-HCl, 100 mM NaCl, 1 mM MgCl2, 0.1 mM EDTA, 0.5 mM DTT-20% glycerol, pH 7.6) the solution was gel filtered on Sephacryl S-300 columns equilibrated with buffer X. The protein peak containing 19S, as revealed by maximal reactivity in the single ELISA described above (AF1 assay after agarose filtration) was verified by non-denaturating gel electrophoresis.

Rabbits were immunized with the purified subunits by 4 doses of 100 μg administrated during one month, resulting in antiserum specific for the 19S proteasome subunit. Catching antibodies specific for 20Sα6 (Enzo Life science) diluted 1:1000 were coated on a Nunc 96-well Maxisorppolyvinyl plate (Sigma-Aldrich) and the polyclonal anti-19S, diluted 1:400, used as detecting antibody. 26S proteasome (Enzo life science) was used as reference and rabbit pre-immune serum as control. A commercial kit (Enzo life science) was used for detection of 20S subunits in blood plasma. This kit also uses the 20Sα6 monoclonal as catching antibody and a polyclonal 20S as detecting antibody.

Western Blot

SDS-polyacrylamide gel electrophoresis (PAGE) and western blot were performed as previously described (Johansson, E., I. Lonnroth, S. Lange, I. Jonson, E. Jennische, and C. Lonnroth, Molecular cloning and expression of a pituitary gland protein modulating intestinal fluid secretion. J Biol Chem, 1995. 270(35): p. 20615-20.). Polyclonal rabbit antibody specific for complement C3c at 1:25000 dilution was used as detecting antibody. The plasma samples were diluted 1:10.

Agarose Incubation.

The agarose Sepharose 6B from GE Healthcare Biosciences was used for achieving in vitro proteasome-C3 complex formation. To a compact gel of agarose, an equal volume of citrate buffer (glucose 0.11 M, tri-sodium-citrate 0.03 M, sodium chloride 0.07 M, pH 6.1) was added. A volume of the gel suspension was added to a test tube, centrifuged at 1500×g for 10 min, the supernatant discarded and two volumes of blood plasma added to give a plasma-gel ratio of 1:3. The plasma-agarose mixture was placed on a blood rocker at 37° C. for 4 hours. The agarose was pelleted by centrifugation and the plasma kept frozen at −20° C. until further testing.

Purification of Proteasome-C3 Complexes

Blood plasma was incubated with agarose as described above and thereafter precipitated with an equal volume of saturated ammonium sulfate. After centrifugation, the precipitate was dissolved in four volumes of binding buffer (25 mM HEPES, 10% glycerol, 5 mM MgCl₂, 1 mM ATP, 1 mM dithiothreitol, pH 7.4) to give a protein concentration of 1 mg/ml. Partial purification of the proteasome-C3 complex was achieved by using a proteasome purification kit BML-PW1075A from Enzo Life Sciences. In short, 100 μg of the dissolved protein was incubated over night at 5° C. with a proteasome binding matrix consisting of agarose-bound anti 20Sβ5. After washing the bound proteasome material with binding buffer three times, 25 μl SDS-PAGE gel loading buffer was added to the matrix and the bound protein eluted at 95° C. for 10 min. nine μl was applied to a western blot gel, and the remaining sample to an SDS-PAGE gel for mass-spectrometric (MS) analysis as described below.

LC-MS/MS and Protein Identification

The coomassie stained gel piece was cut out and protein digested with trypsin. Briefly, the gel piece was washed three times in 25 mM NH₄HCO₃ in 50% CH₃CN and one time in 25 mM NH₄HCO₃ in 50% CH₃OH. The gel piece was dried in a vacuum centrifuge and incubated with trypsin (Promega, Madison, Wis., USA) at 10 ng/μl in 25 mM NH₄HCO₃ at 37° C. overnight. Peptides were extracted by first adding 75% CH₃CN/2% TFA, followed by an extraction with 50% CH₃CN/0.2% TFA. The pooled extracts were evaporated to dryness in a vacuum centrifuge and reconstituted in 18 μl of 0.2% HCOOH prior to MS analysis. The nanoflow LC-MS/MS was performed on a hybrid linear ion trap-FT-ICR mass spectrometer equipped with a 7T ICR magnet (LTQ-FT, Thermo Electron, Bremen, Germany). For protein identification, the minimum criteria was set to at least two tryptic peptides matched at or above the 95% level of confidence given by MASCOT.

Detection of Complement I and H

Commercial ELISA kits for determination of complement I and H concentrations in plasma were used. The complement I kit from AssayPro, USA (www.assaypro.com) allowed us to determine complement I concentration in the range 0.4-20 μg/ml; the complement H kit from Blue Gene (www.bluegene.cc) determined H concentration in the range 5-100 ng/ml.

Virus HSV-1 2762, a clinical isolate from a brain biopsy of a 58-year-old male patient who presented with focal encephalitis which later turned out to be fatal (Bergstrom et al. 1990), was used for infection of rats. The virus has been shown to be highly neurovirulent in animal models (Bergstrom et al. 1990). Isolation of the virus from the brain biopsy was approved in 1981 by the ethics committees at the universities of Gothenburg, Linköping, Lund, Umeå and Uppsala at the Karolinska Institute, for a Swedish multicentre study on antiviral treatment in HSE (Skoldenberg et al. 1984) conducted according to the principles expressed in the Helsinki declaration. Virus stocks of HSV-1 strain 2762 were prepared from low passages.

Animals

Male Sprague-Dawley (SD) rats, body weight 250±20 g, (Harlan Laboratories, Boxmeer, The Netherlands) were used (infected rats n=49, control rats n=11). The regional Ethical Committee on Animal Experiments approved the test protocol, and all experiments were performed in accordance with the EC Directive 86/609/EEC guidelines for animal experiments. The rats were allowed a week for general adaptation before the start of the experiments, and they had constantly free access to pelleted food and water. The temperature and air ventilation in the animal quarters were monitored according to standard procedures; a 12-h light cycle was used and the air was exchanged 17 times per hour.

Intranasal Injection

The encephalitogenic infection in rats was achieved after intranasal instillation in the right nostril with HSV-1, with a volume of 25 μl (total dose given=1.1×10⁴ plaque forming units (pfu)), as described previously (Jennische et al. 2008). This instillation was adapted from an inoculation method described by RT Johnson (Johnson 1964). The infective dose was always placed in the right nostril while the rat was under deep isoflurane (Forane, Baxter, Deerfield, Ill.) anesthesia. The rats were sacrificed day 4 post-infection. For ethical reasons, the extent of the experiment could not exceed day 6 p.i., as all animals by then were symptomatic.

Results

Proteasome Concentration in Blood Plasma

Two different immunoassays were used to determine the proteasome concentration in blood plasma. The commercial kit based on antibodies specific for the 20S subunit gave a concentration of 0.60±0.07 μg/ml in normal plasma, using 20S proteasomes as standard. With the herein disclosed test using monoclonal 20S and 19S antibodies in combination, the corresponding value was 0.41±0.03 μg/ml. In both these tests monoclonal anti-20Sα6 was used as catching antibody. When anti-AF1 (AF monoclonal antibody (mAb), 3H8 derived from the hybridoma cell culture 2341 (=3H8B3), which is deposited at the DSMZ under the deposition number DSM ACC3271), recognizing antisecretory peptide sequences, was used for catching, no binding to intact 26S proteasomes was obtained.

Rise of Agarose-Separated AF1 Concentration after Intake of Processed Cereals

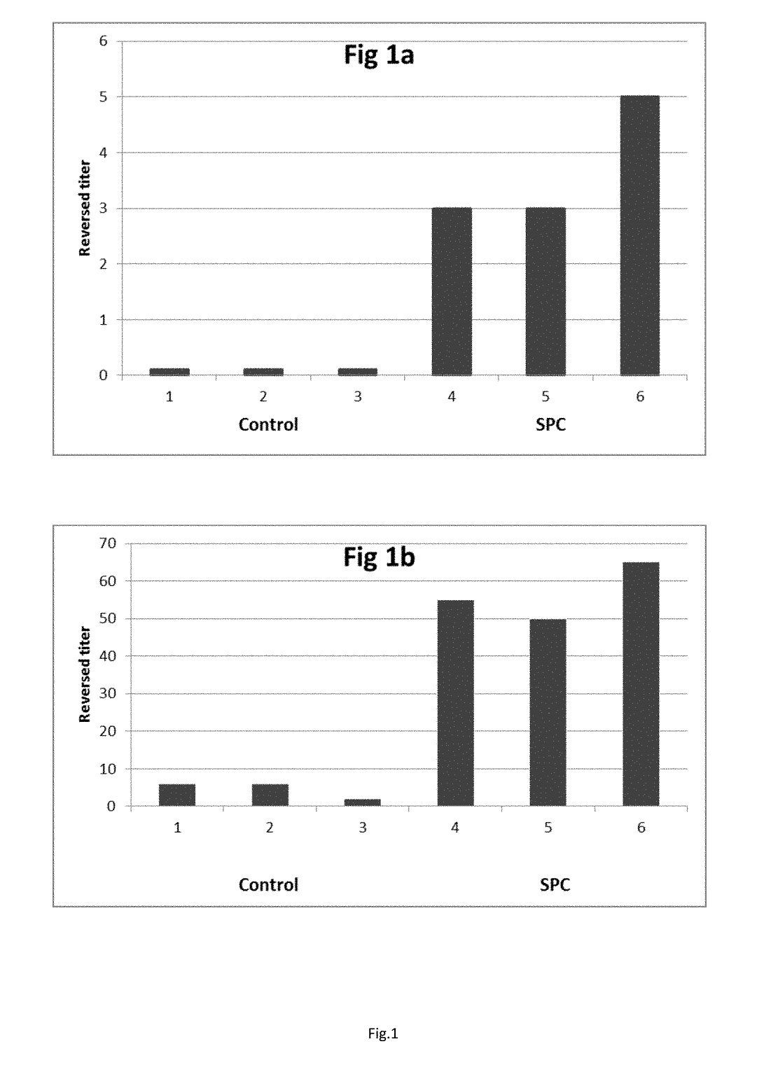

Previous studies have shown that AF1, as well as C3c, binds to agarose gel after consumption of specially processed cereals (SPC). This was confirmed in the present study of agarose-filtered plasma from six volunteers. FIG. 1a shows no apparent AF1 value in the control persons 1-3, whereas persons 4-6 had a significantly higher AF1 titer after eating SPC more than two months.

Induction of AF1-Reactive Proteasome/Complement C3 Complexes by Processed Cereals

In order to test if there was a reaction between proteasomes and complement factor C3 in blood, two new ELISA assays were developed: monoclonal antibodies specific for proteasome components AF1 (AF monoclonal antibody (mAb), 3H8 derived from the hybridoma cell culture 2341 (=3H8B3), which is deposited at the DSMZ under the deposition number DSM ACC3271) were employed as catching antibodies, while polyclonal antibodies specific for C3c was used as detecting antibodies. This enabled measuring the attachment of proteasomes and C3 directly in plasma. As seen in FIG. 1b, the three samples from test persons 4, 5 and 6 had an 8-11 times higher titer than those from the control persons 1, 2 and 3. Due to its high sensitivity, only a small sample of 100 μl of blood is sufficient to perform the test.

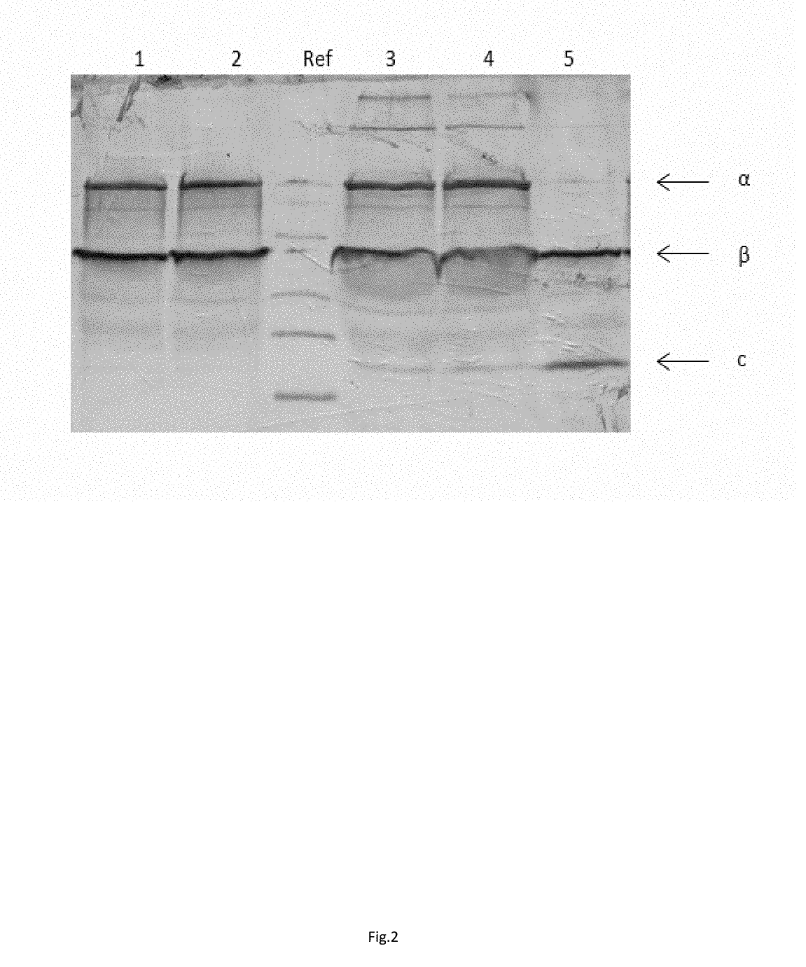

Induction AF1-exposed of proteasome/complement C3 complexes by agarose gel In order to test the influence of agarose on the plasma proteasome and factor C3, the plasma was incubated with agarose gel for 4 hours at 37° C. A substantial increase in the aggregation of AF1-exposed proteasomes and C3 by agarose was observed in all six test persons (Table 1). When analyzed by western blot, a concomitant splitting of C3 was evident (FIG. 2, row 5). The a band was hydrolyzed into the c band, which is typical for a conversion of C3 into C3c. In addition, when two of the control plasmas were compared to the SPC test plasmas (FIG. 2, row 1-4), a difference in the peptide pattern was also observed. The SPC plasma had extra bands both in the c region and in a region of molecular weights exceeding those of C3.

Partial Purification of AF1-Exposed Proteasome/C3 Complex

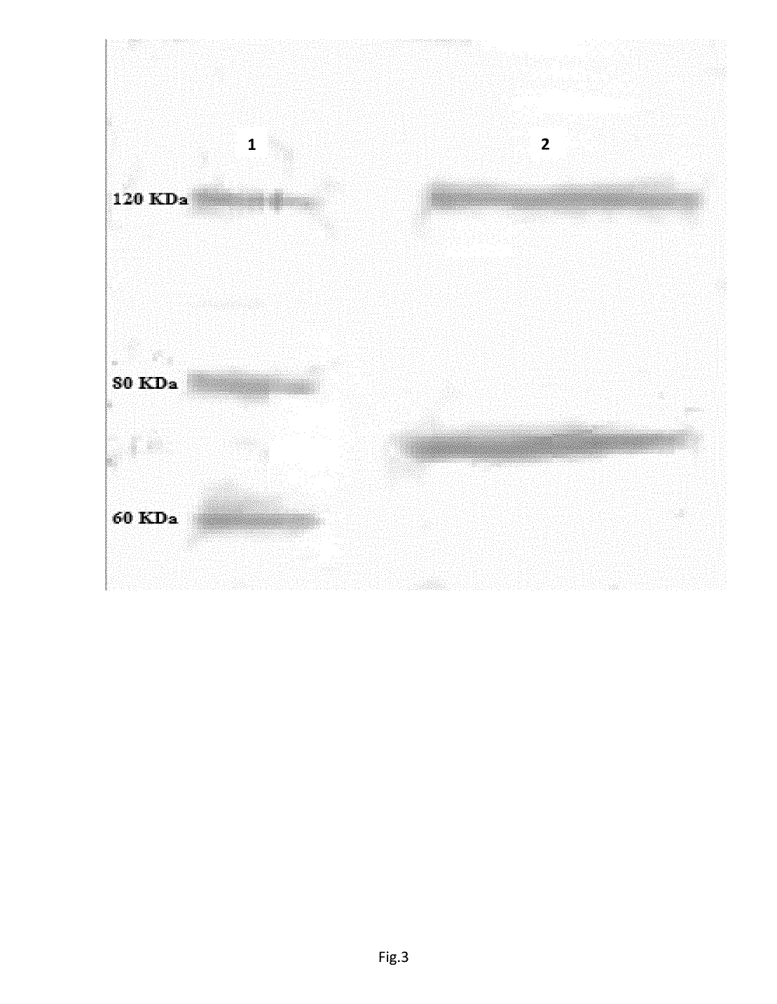

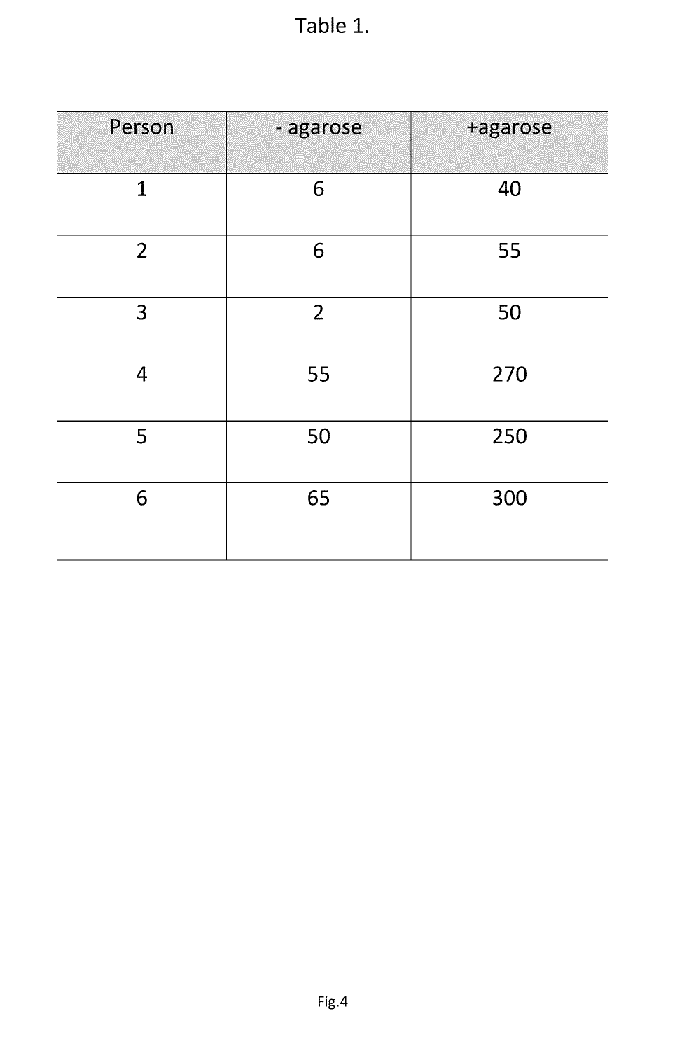

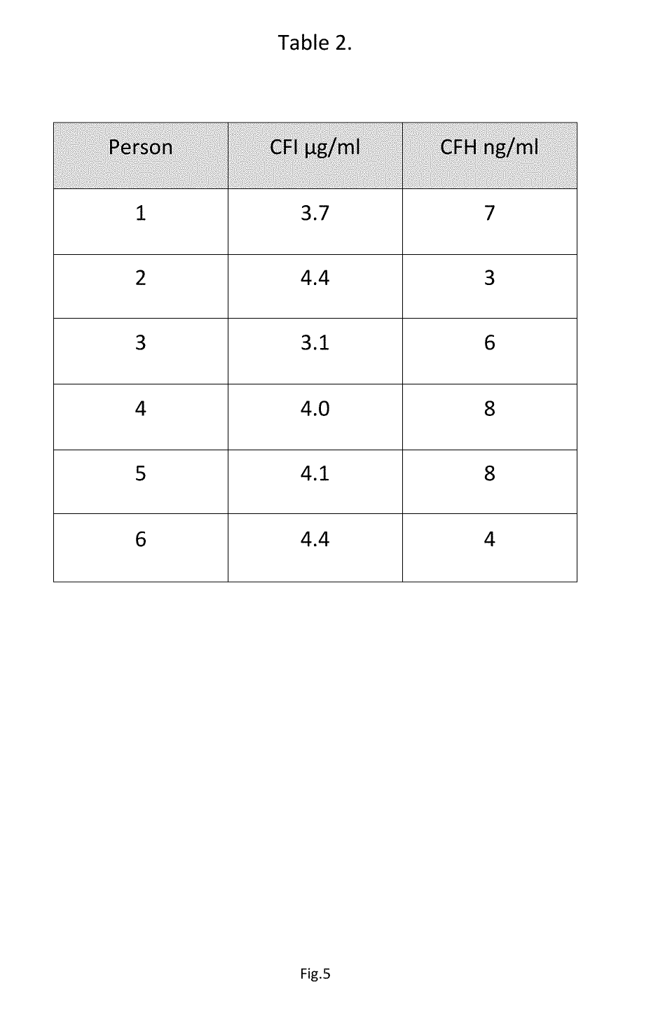

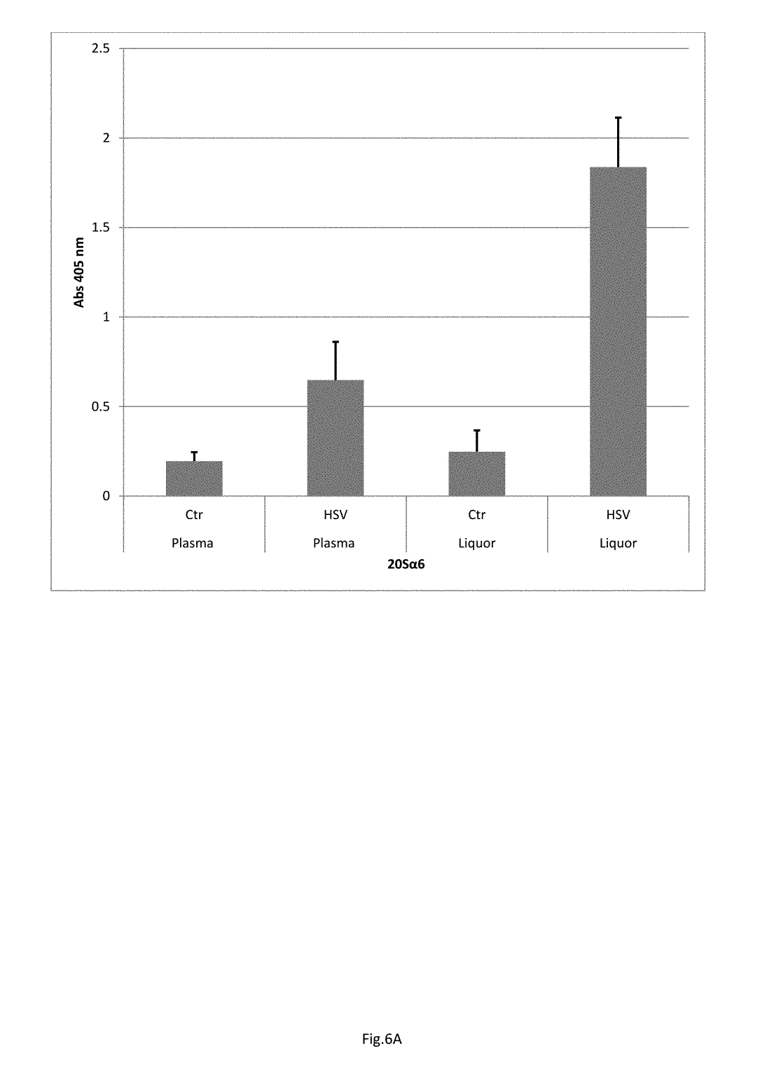

A partial purification of the proteasome-C3 complex was achieved by affinity to a matrix-bound proteasome antibody. Agarose stimulated and ammonium sulfate precipitated blood plasma was attached to the matrix, which after washing was shown by western blot to contain C3 as well as proteasomes (FIG. 3). The content of C3 was confirmed by cutting out coomassie-stained gel bands and running trypsin-digested bands in MS/MS; the α- and β-bands of C3 appeared as the primary bands at positions corresponding to those in western blot.

Blood Levels of Complement H and I

The formation of C3c is achieved by the complement factors I and H. It was therefore tested for these complement factors in the six plasma samples. As shown in Table 2 there was no difference in the level of these components in control versus test plasma.

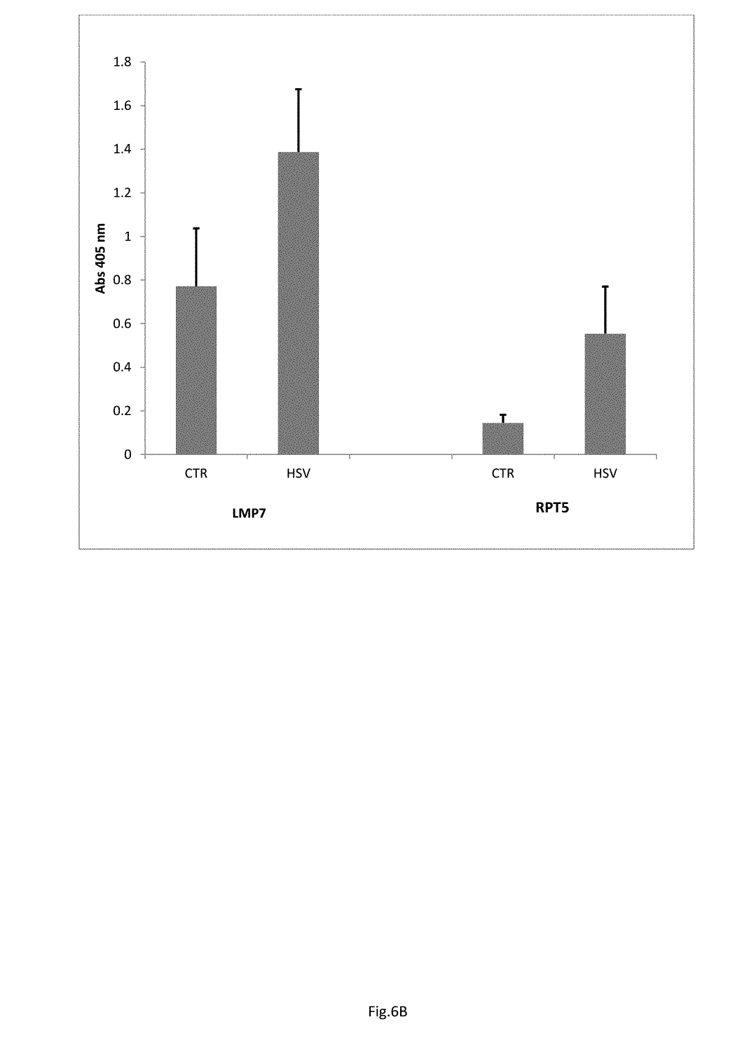

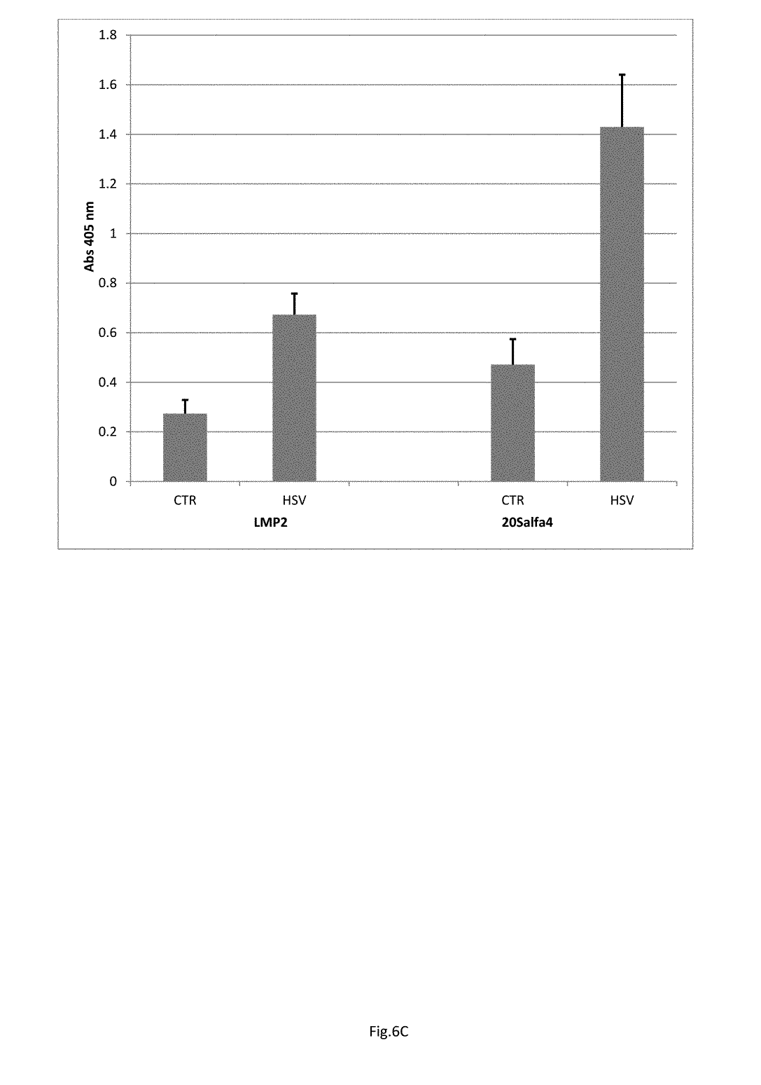

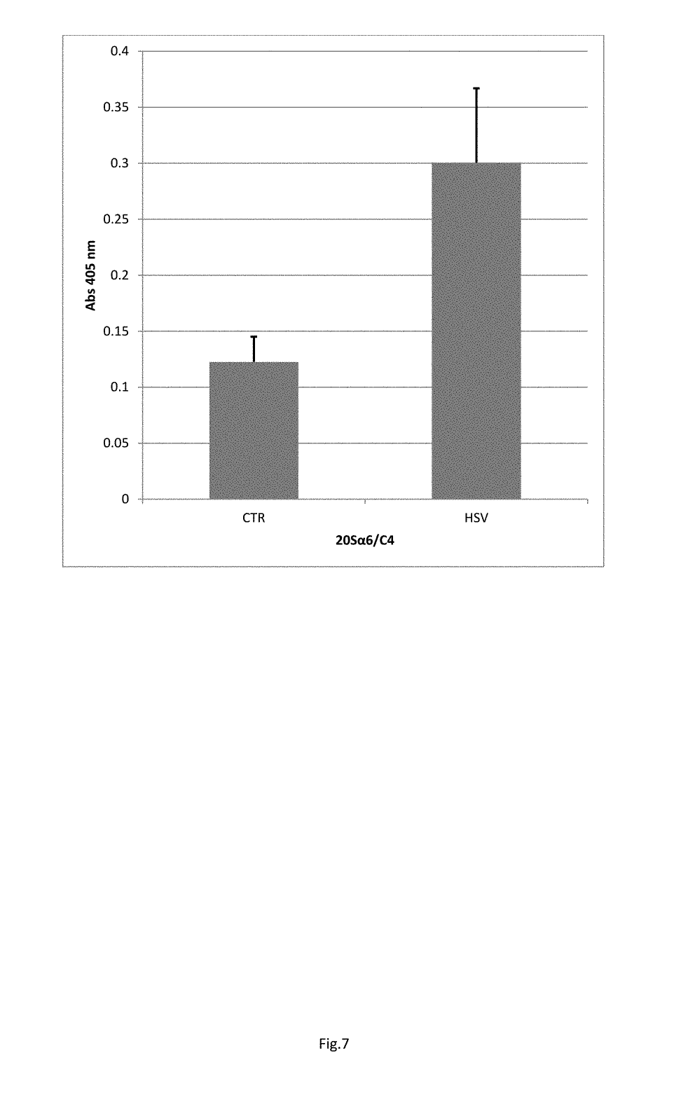

Induction of proteasome/complement complexes by herpes simplex virus 2 in rat The impact of herpes simplex virus 1 (HSV1) on proteasome/complement factor complex formation was tested in a rat model. The rats were tested after 4 days challenge intranassally when they started to show symptoms of neurological dysfunction, i.e. repetitive, stereotypic movements and motor instability. A number of proteasomal monoclonal antibodies were tested as catching antibody and antibodies against complement factor 3 and 4 as detecting antibody. As seen in FIG. 6, a significant rise of proteasome/C3 complex was noted in blood and liquor after infection. Also a significant rise of proteasome/C4 complexes was noted after HSV1 infection (FIG. 7).

However, AF1 antibodies (AF monoclonal antibody (mAb), 3H8 derived from the hybridoma cell culture 2341 (=3H8B3), which is deposited at the DSMZ under the deposition number DSM ACC3271) did not react with the HSV1 induced complexes (not shown) in which the AF1 epitopes remained hidden like in intact 26S proteasomes.

### EXAMPLE 2

Investigation of Changes in Proteome of Plasma after SPC Diet

Objective: Antisecretory Factor (AF) inhibits pathological fluid secretion and inflammation. After challenge with bacterial enterotoxins, induction of an altered form with increased AF-activity follows. This form of AF is designated “active AF” and is also increased after intake of Specially Processed Cereals, SPC. The molecular events that influence transition from inactive to active form have remained vague. The aim of this study was to investigate changes in proteome of plasma after SPC diet.

Research Methods & Procedures:

Plasma samples were taken from human volunteers before and after intake of SPC for at least four weeks. Changes in plasma after agarose-purification were analyzed by using 2D DIGE and LC-MS/MS followed by Western blot and by ELISA. The time course for up-regulated proteins was thereafter studied in plasma after 4-, 7-days of SPC diet and 1 week after termination.

Results. The level of complement factor C3c was significantly up-regulated (p=0.0039) in agarose-purified human plasma after six weeks of SPC diet. The increased C3c level was associated to the raised level of AF-activity. The regulator Factor H and C4 were also up-regulated in agarose-purified plasma after SPC-intake. AF induction reached significance first after 7 days (p=0.0011), compared to 4 days of SPC-intake for significant increase of C3 (p=0.0077) but not for AF-activity (p=0.31) at day four. The up-regulated C3c was only detected in plasma after the agarose-purification.

Conclusion: Specific confirmation changes in the complement system seem to be involved in the mechanisms behind increased AF-activity in plasma in response to stimulation.

The present study includes proteomic analysis, 2 dimensional fluorescence difference gel electrophoresis (2D DIGE) and nanoflow liquid chromatography-tandem mass spectrometry (LC-MS/MS), to identify up-regulated expressed proteins which suggest involvement of the complement system in the mechanism behind increased AF-activity in human agarose-purified plasma as response to SPC stimulation.

Material and Methods

First Part of the Study: Proteomic Analysis

Preparation of Plasma-AF

AF was purified from plasma using affinity-chromatography (10). In brief, after passage of the plasma through a 0.9 cm Ø agarose column (Sepharose 6B, GE Healthcare Bio-sciences AB) and washing in phosphate buffered saline (PBS), the agarose absorbed AF was eluted with 1 M methyl-α-D-glucoside and stored at −20° C. until analysis.

2D Gel Electrophoresis

Agarose-purified plasma samples drawn from three individuals, the samples were collected before and after four weeks of SPC-intake. Thereafter, analyzing with isoelectric focusing (IEF) and second dimension (2D) electrophoresis followed. The samples were purified with the 2D Clean-Up kit (Amersham Biosciences) before suspension in 2D sample solution. IEF was performed using IPGphor system (Amersham Biosciences) following the manufacturer's instructions. Immobiline dry stripgels (7 cm pH 3-10) were hydrated overnight in 125 μl of solution containing 2% CHAPS (w/v), 0.3% DTT (w/v), 8 M urea, 0.5% carrier ampholytes solution (v/v), and 100 μl of sample plasma in the 2D sample solution. IEF was performed for 30 min at 500 V, 30 min 1000 V, and 1.5 hat 5000 V. Prior to 2D separation, immobiline dry strip gel strips were soaked for 15 min in 2.5 ml equilibration buffer (6 M urea, 30% glycerol, 2% SDS, 1% DTT, and 0.001% bromphenol blue in 1.5 M Tris-HCL buffer, pH 8.8). The immobiline dry strip gel strips were placed on 2D well in 10% Tris-Glycine gels (Invitrogen) and were run according to the manufacturer's instructions. The gels were stained with SilverQuest™ Staining kit (Invitrogen) or blotted onto PVDF membranes. The membranes were blocked with 1% bovine serum albumin (BSA) in PBS at 4° C. for 16 h. The primary polyclonal anti-C3c (diluted 1/25 000) from Dako (Glostrup, Denmark) was then added to the membranes and incubated for 1.5 h. The blots were developed with alkaline phosphatase (AP)-conjugated goat anti-rabbit IgG (Jackson ImmunoResearch Europe Ltd.), followed by 5-bromo-4-chloro-3-indolylphosphate and 4-nitro blue tetrazolium (Roche Diagnostics).

Protein Digestion and Peptide Extraction

The silver stained gel piece was cut out and destained according to the manufacturer's protocol (SilverQuest™ Silver Staining Kit, Invitrogen). After the silver destaining the method for in-gel protein digestion with trypsin described by Shevchenko et al (20) was applied with some minor modifications. Briefly, the gel piece was washed three times in 25 mM NH₄HCO₃ in 50% CH₃CN and one time in 25 mM NH₄HCO₃ in 50% CH₃OH. The gel piece was dried in a vacuum centrifuge and incubated with trypsin (Promega, Madison, Wis., USA) 10 ng/μl in 25 mM NH₄HCO₃ at 37° C. overnight. Peptides were extracted first by adding 75% CH₃CN/2% zTFA and followed by another extraction in 50% CH₃CN/0.2% TFA. The pooled extracts were evaporated to dryness in a vacuum centrifuge and reconstituted in 18 μl of 0.2% HCOOH prior to MS analysis.

Nanoflow LC-MS/MS and Protein Identification

A three-microliter sample injection was made with an HTC-PAL autosampler (CTC Analytics AG, Zwingen, Switzerland) connected to an Agilent 1100 binary pump (Agilent Technologies, Palo Alto, Calif., USA). The peptides were trapped on a precolumn (45×0.075 mm ID) and separated on a reversed phase column (200×0.050 mm ID). Both columns were packed in-house with 3 μm Reprosil-Pur C₁₈-AQ particles. The flow through the analytical column was reduced by a split to approximately 100 nl/min. A 37 min gradient 10-50% CH3CN in 0.2% COOH was used for separation of the peptides with a hold at 80% B for another 5 min. The nanoflow LC-MS/MS was performed on a hybrid linear ion trap-FT-ICR mass spectrometer equipped with a 7T ICR magnet (LTQ-FT, Thermo Electron, Bremen,

Germany). The spectrometer was operated in data-dependent mode, automatically switching to MS/MS mode. MS-spectra were acquired in the FT-ICR, while MS/MS-spectra were acquired in the LTQ-trap. For each scan of FT-ICR, the six most intense doubly or triply charged ions were sequentially fragmented in the linear ion trap by collision induced dissociation. The MS raw data file was used for identification using Proteome Discoverer version 1.4 (Thermo Fisher Scientific, Inc., Waltham, Mass., USA). One database search was performed with the MASCOT search engine, version 2.3 (Matrix Science LTD., London, United Kingdom) using the Swissprot Database (Swiss Institute of Bioinformatics, Switzerland) downloaded in July 2014 (containing 20265 sequences after taxonomy). The search parameters were set to: Taxonomy human, MS accuracy 5 ppm, MS/MS accuracy 0.5 Da, one missed cleavage by trypsin allowed, fixed propionamide modification of cysteine and oxidation of methionine as a variable modification.

For protein identification the minimum criteria was set to at least two tryptic peptides matched at or above the 95% level of confidence given by MASCOT.

Second Part of the Study: Conformation of Complement

Subjects and Study Design

Three healthy volunteers took part in the second part of the study. The Human Ethics

Committee of the University of Gothenburg approved the experimental procedures (Dnr. 037-14). All volunteers ingested SPC (AS-Faktor AB, Stockholm, Sweden), in a dose of 1 g per kg body weight and day. SPC intake was divided in two servings per 12 hours for a period of three weeks. Plasma samples were drawn from each volunteer before start and after 4- and 7-days. One week after termination of the SPC diet the last sample was drawn. After centrifugation at 1000×g for 5 min, the plasma samples were mixed with Alsevers citrate buffer (1/2). AF was thereafter purified as described above in section “Preparation of plasma-AF”.

ELISA Test of Immunogenic AF Activity

The content of AF in plasma was assayed in an enzyme-linked immunosorbent assay (ELISA) as described previously. Maxisorp microtiter plates (Nunc) were coated with the affinity purified plasma, diluted 1/2 thereafter serial diluted 1/3, and then incubated overnight at 4° C. After blocking with 0.2% BSA in PBS for 45 min at 37° C., plates were washed with PBS+0.05% Tween 20 (PBS-T). The AF monoclonal antibody (mAb), 3H8 (AF monoclonal antibody (mAb), 3H8 derived from the hybridoma cell culture 2341 (=3H8B3), which is deposited at the DSMZ under the deposition number DSM ACC3271), (diluted 1/100 in PBS+0.05% Tween 20+0.2% BSA) was added and the plates were incubated for 2 h at room temperature (RT). After washing, AP-conjugated goat anti-mouse immunoglobulin IgM (Jackson immunoResearch Europe Ltd.) was added for 1 h at RT. After washing, the substrate 4-Nitrophenyl phosphate (Sigma-Aldrich Sweden AB) in diethanolamine buffer (pH 9.8) with 1 mM MgCl₂ was added to the plates and the bound enzyme was revealed by reading absorbance at 405 nm. Monoclonal culture medium was used as background and the values were deducted to give the net absorbance.

ELISA Detection of Complement Factors

To study the of effect on the complement system after SPC-intake, the level of seven complement factors in the affinity purified human plasma samples was determined by ELISA with the same procedure as described in section “ELISA test of immunogenic AF activity”. As the detecting antibodies we were using rabbit anti-C3c (diluted 1/2000) and anti-C4c (diluted 1/1000) from Dako (Glostrup, Denmark). Mouse anti-C2 and anti-C5 (diluted 1/200), goat anti-C1q and anti-C6 (diluted 1/200) and rabbit anti-Factor H (diluted 1/200) were purchased from Santa Cruz Biotechnology (Heidelberg, Germany). AP-conjugated second antibodies of goat anti-rabbit IgG, goat anti-mouse IgG or donkey anti-goat (Jackson ImmunoResearch Europe Ltd.) were then applied (diluted 1/10 000). Pre-immune serum was used as background and the values were deducted to give the net absorbance.

SDS-Polyacrylamide Gel Electrophoresis (SDS-PAGE) and Western Blot

The content of C3c was determined in plasma samples drawn before and after the SPC-intake, C3c was also analyzed before and after agarose-purification of plasma, by SDS-PAGE followed by immunoblotting onto nitrocellulose membrane. The subsequent incubations were the same as for the Western blot assay described above, in section “2D gel electrophoresis”.

Statistical Analysis

Data are presented as mean±standard error of the mean (SEM). GraphPad Prism 5.0 software (Graphpad Software, Inc., San Diago, Calif.) was used to compare the samples before and after SPC-intake. Statistical significance was determined by using one-way ANOVA and t-test. A p-value ≦0.05 was considered significant.

Results

2D Analysis of Proteins in Agarose-Purified Plasma after Induction of AF

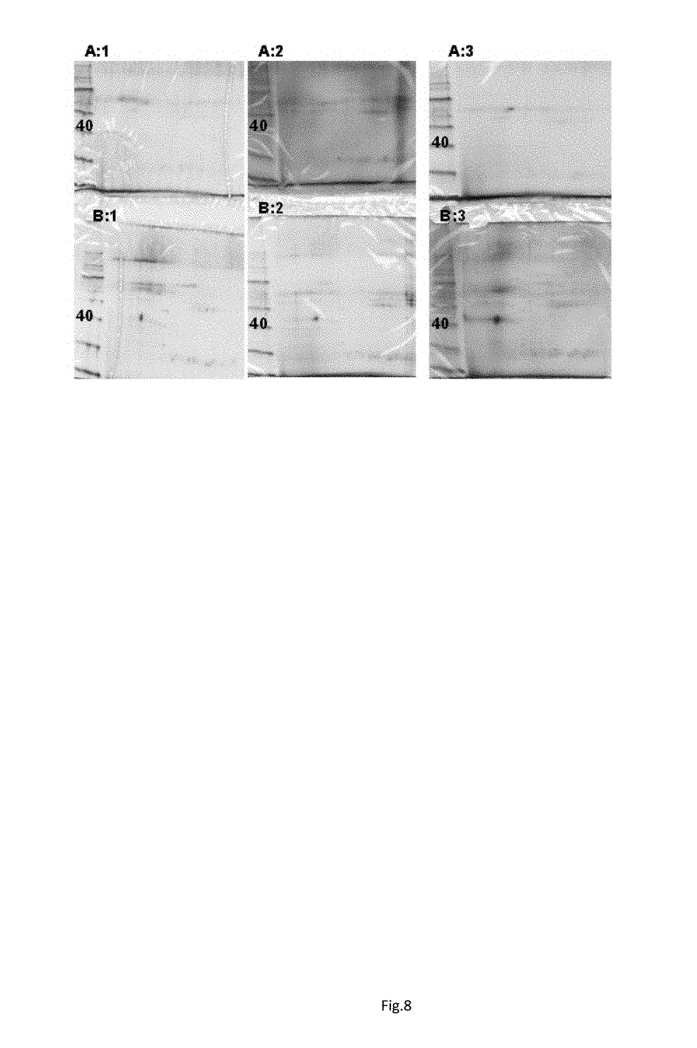

2D separation was performed in the pH range of 3-10 on purified plasma samples taken from three individuals before and after 4 weeks of intake of SPC which had resulted in increased AF activity in these plasma samples. By comparing the protein expression levels between the two groups of samples, a distinct spot on the silver stained gel was apparent in the region to isoelectric point of 4.8 and molecular weight of approximately 43 kDa after induction of AF (FIG. 8).

MS Analysis

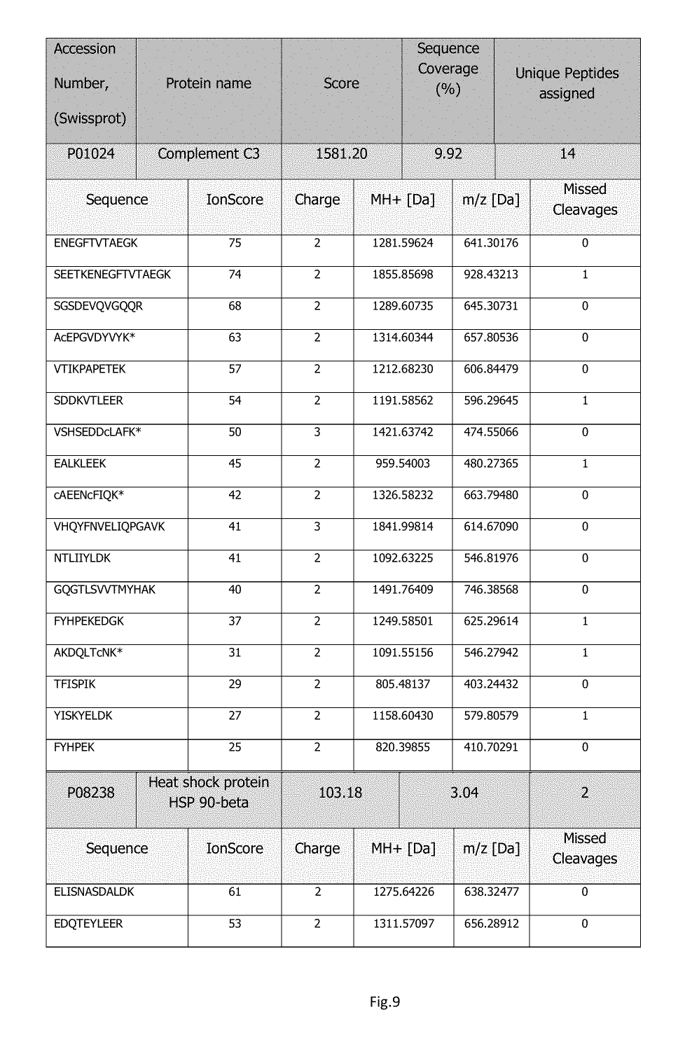

Identification of the chosen plasma protein spot from the gel was performed by 2D DIGE followed by LC-MS/MS and the result is summarized in Table 3 (FIG. 9). After removal of apparent contamination proteins, such as keratin and trypsin, two proteins were remaining in the identity list. They included Complement C3 precursor (score 1581.20) and Heat shock protein HSP 90-beta (score 103.18). As the HSP was of bovine source with only 2 assigned peptides, the human Complement C3 precursor was the most relevant identity in the agarose-purified plasma, with 14 unique peptides demonstrating >95% confidence. The peptides were all matching aa in the C-terminal part of the C3 precursor sequence, starting from aa 1331 to aa 1599 of the 1663 full-length protein. As the selected protein spot was in the region of approximately 43 kDa and μl of 4.8, the analyzed protein is most probably the split product of C3 into C3c a chain fragment 2.

Validation of Proteomic Identify C3c by Western Blot Analysis and ELISA

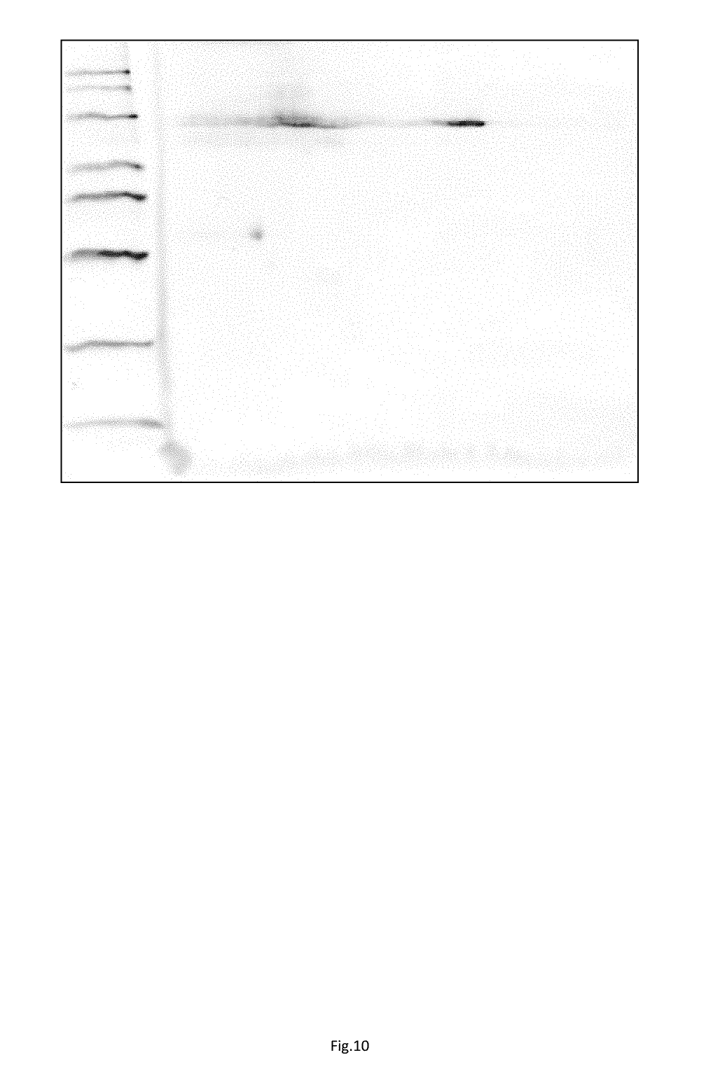

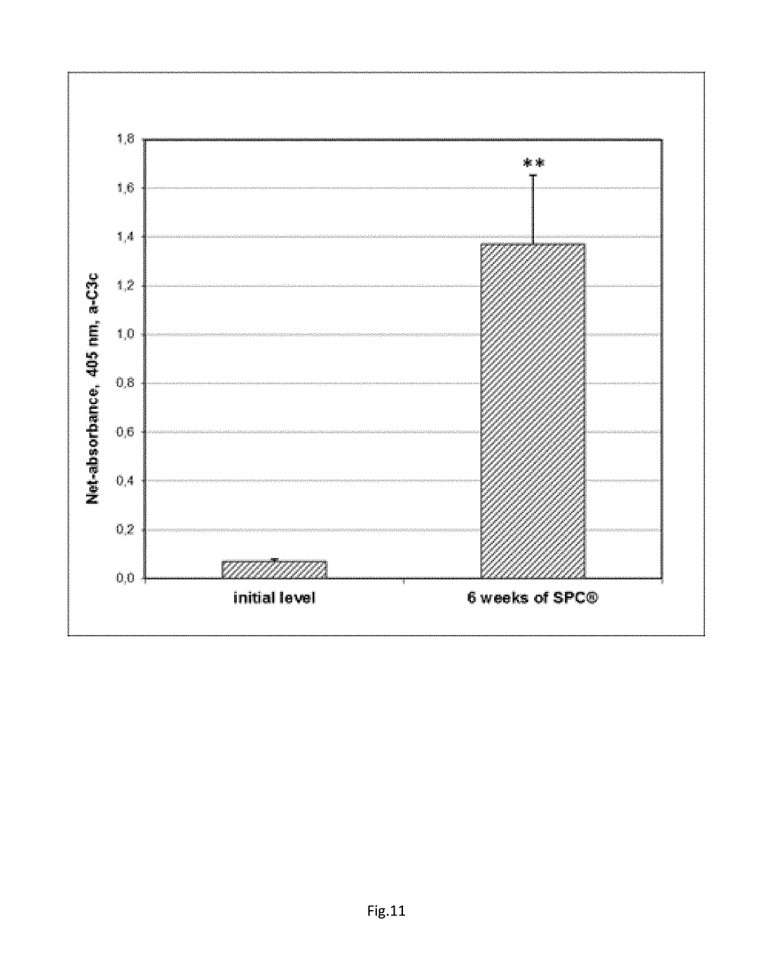

To confirm the identified spot from the silver stained 2D gel, the expression of C3c was analysed by Western blot using the C3c antibody. As shown in FIG. 10, the antibody reacted to agarose-purified plasma, drawn after SPC diet, with a dot at isoelectric point of 4.8 and a molecular mass of 43 kDa indicating the expected C3c a chain had been cleaved from C3. The antibody detected also the 75 kDa β band of C3 at isoelectric point of 6.8 together with a smear staining, indicating chemical modifications of the protein. The up-regulation of C3c in the human agarose-purified plasma samples was also confirmed by ELISA determination. As seen in FIG. 11, a significant increase of C3c was detected in the AF-induced samples after 6 weeks of

SPC-intake (p=0.0039).

AF-Activity in Plasma after Intake of SPC

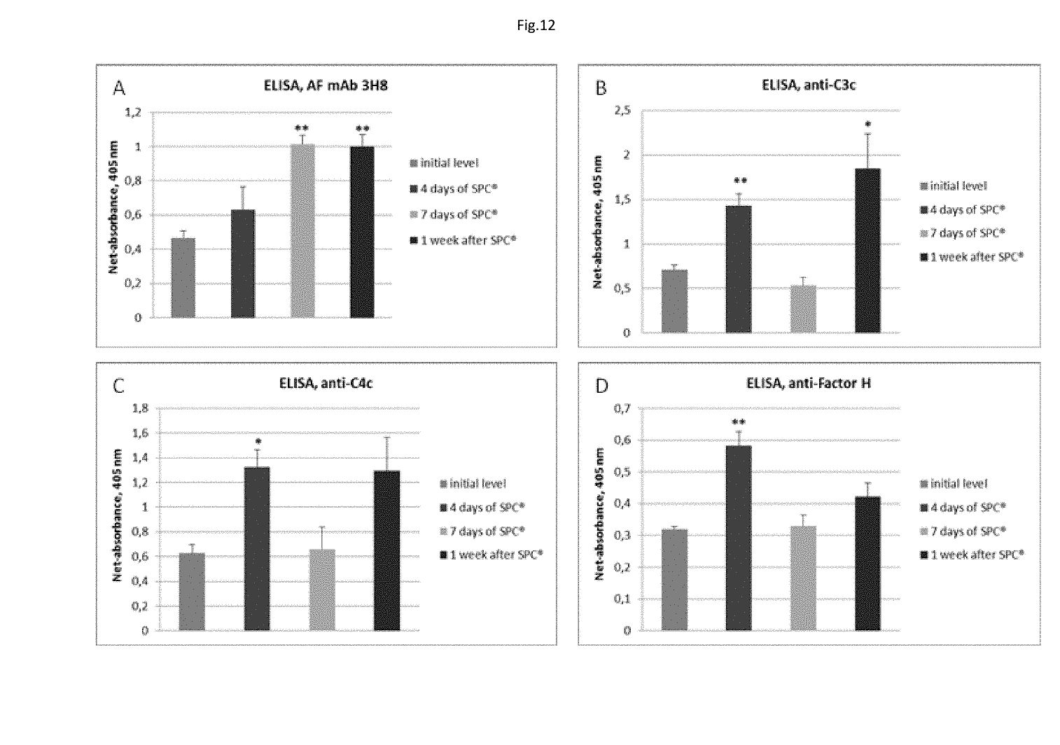

In the second part of the study we continued to analyze the results after SPC-diet. To evaluate the effect of SPC stimulation, the level of AF activity in agarose-purified plasma was determined with AF mAb 3H8 (AF monoclonal antibody (mAb), 3H8 derived from the cell culture 2341 (=3H8B3)=DSM ACC3271). As shown in FIG. 12A, compared with the control plasma, the absorbance after SPC intake was increased successively in the assay. A significant increase in plasma levels of active AF was induced after 7 days of SPC (p=0.0011). The AF activity continued to be significant increased even 1 week after termination of the diet (p=0.0023).

ELISA Determination of C3c and for other Complement Factors

In order to investigate additional complement factors, besides C3c, ELISA were performed on agarose-purified plasma. Together with C3c, seven complement factors were tested to determine the influence on the complement system after intake of SPC. Three of the factors, C3c, C4c and Factor H showed an increased level in agarose-purified plasma after AF induction (FIG. 12B, C, D). Whereas AF induction reached significance after 7 days of SPC diet (FIG. 12A), the significant increase in plasma levels of complement factors were already induced after 4 days (p=0.0077 for C3c, p=0.011 for C4c and p=0.0042 for Factor H compared to p=0.31 for AF). The mean value of C3c continued to be significantly increased also 1 week after SPC termination (p=0.0475).

No differences were seen in ELISA testing C1q, C2, C5 or C6 in any of the samples.

Western Blot Analysis on Direct Plasma and Agarose-Purified Plasma

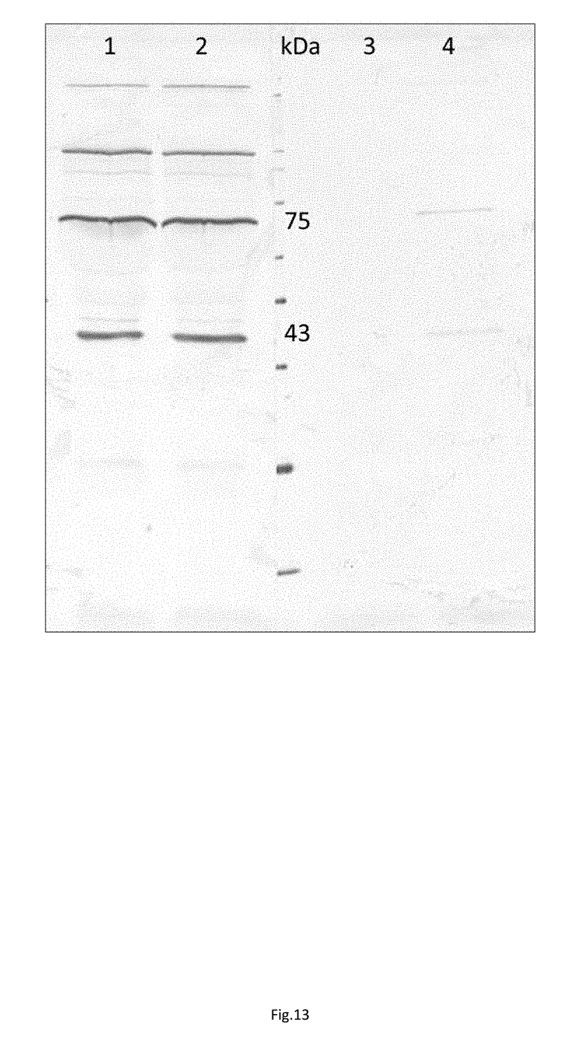

Plasma samples from the study, before and after agarose-purification, were tested by Western blot using antibody against complement factor C3c. The samples also included plasma taken before and after SPC-intake with determined increase of AF-activity. As seen in FIG. 13, lane 1 and 2, there were no visible differences in the anti-C3c reaction on the separated direct plasma samples. The antibody detected several protein-bands originate from the Complement C3 precursor in the same way, despite the known AF induction. In contrast, only in the sample taken after SPC diet, the antibody against C3c reacted on the agarose-purified plasma detecting the two chains, C3c α and the β band of C3 (FIG. 13, lane 4).

Discussion

Activation of endogenous AF occurs as a natural response to intestinal exposure of enterotoxins or can be mimicked by intake of certain food components, i.e. SPC. In search of the molecular events behind the activation steps of AF, it was investigated agarose-purified plasma after SPC-intake using a proteomic approach; 2D DIGE with following identification by LC-MS/MS of up-regulated protein expression. Analysis of agarose-purified plasma proteins, before and after intake of SPC diet revealed a spot in the AF induced sample identified as complement factor C3c a chain fragment 2. The given result of increased C3c after SPC was confirmed in plasma samples by showing a specific antibody-reaction in ELISA and in Western blot. The present inventors have previously shown that induced AF levels are related to the dosage of SPC as well as to the time period of intak. In this study, it was investigated the influence of SPC on seven complement system proteins. A significant increased binding to agarose of complement proteins C3c, Factor H and C4c, were seen already after 4 days of SPC-intake. A tendency of activation of AF could be determined after 4 days, but a significant increased level was detected 7 days after the SPC diet. Previous studies have demonstrated AF increase 2 weeks after the diet. The difference might be due to different study design with lack of samples taken a few days after start of SPC-intake.

The results indicate that intake of SPC inactivate factor C3 and C4 of the complement system. Several factors in this system are crucial components of innate and adaptive immunity, and might consequently explain the noticeable anti-inflammatory reaction during the AF activation. The complement system has also been proposed to be involved in alternative functions, indicating that it has important roles in diverse biologic processes that are divergent from its inflammatory role. E.g. normal pregnancy is associated with increased plasma complement components.

C3 plays a central role in complement system through its activation, by undergoing large conformational changes to form activated fragments that by sequential proteolysis in the end form deactivation into C3c, the physiological down-regulation product of C3 (23).

The split of C3 into C3c has previously been shown to involve the proteolytic complement Factor I with Factor H as a cofactor. The early increase of Factor H demonstrated in this study can be seen as a down-regulation of the complement system. This reaction would ultimately protect host cells from damage resulting from unrestrained complement activation. Concomitantly, C4 is down regulated by Factor I and H to C4c.

In this study complement C3c together with AF were identified when analyzing plasma that was firstly affinity purified on agarose and eluted with a-methylglucoside. The present inventors were not able to detect any C3c increase by Western blot in direct plasma after SPC-intake.

Conclusion

This study provides new fundamentals describing the mechanism behind activation of AF. 2D separated plasma after SPC diet, demonstrated an early up-regulation of complement C3c, preceding increase of activated AF. Conclusively, specific conformation changes in the complement system might therefor be the first step during AF activation.

### REFERENCES

1. WO 97/08202;

2. WO 05/030246

3. WO 97/08202

4. WO 97/08202

5. WO 98/21978

6. WO 00/038535.

7. WO 05/030246

8. WO 07/126364

9. WO 07/126363

10. WO 07/126365

11. WO 2010/093324

12. Lange et al., 2003; Food-induced antisecretory factor activity is correlated with small bowel length in patients with intestinal resections. APMIS. 2003 October; 111(10):985-8.

13. Laurenius et al., 2003; Antisecretory factor counteracts secretory diarrhoea of endocrine origin. Clin Nutr. 2003 Dec; 22(6):549-52.

14. Jennische et al., 2006; Immunohistochemical staining patterns using epitope-specific antibodies indicate conformation variants of antisecretory factor/S5a in the CNS. APMIS. 2006 July-August; 114(7-8):529-38.

15. Tomko, R. J., Jr. and M. Hochstrasser, Molecular architecture and assembly of the eukaryotic proteasome. Annu Rev Biochem, 2013. 82: p. 415-45.

16. Sixt, S. U. and B. Dahlmann, Extracellular, circulating proteasomes and ubiquitin-incidence and relevance. Biochim Biophys Acta, 2008. 1782(12): p. 817-23.

17. Johansson, E., I. Lonnroth, S. Lange, I. Jonson, E. Jennische, and C. Lonnroth, Molecular cloning and expression of a pituitary gland protein modulating intestinal fluid secretion. J Biol Chem, 1995. 270(35): p. 20615-20.

18. Bjorck, S., I. Bosaeus, E. Ek, E. Jennische, I. Lonnroth, E. Johansson, and S. Lange, Food induced stimulation of the antisecretory factor can improve symptoms in human inflammatory bowel disease: a study of a concept. Gut, 2000. 46(6): p. 824-9.

19. Lange, S. and I. Lonnroth, The antisecretory factor: synthesis, anatomical and cellular distribution, and biological action in experimental and clinical studies. Int Rev Cytol, 2001. 210: p. 39-75.

20. Johansson, E., I. Lonnroth, I. Jonson, S. Lange, and E. Jennische, Development of monoclonal antibodies for detection of Antisecretory Factor activity in human plasma. J Immunol Methods, 2009. 342(1-2): p. 64-70.

21. Johansson, E., M. Al-Olama, H. A. Hansson, S. Lange, and E. Jennische, Diet-induced antisecretory factor prevents intracranial hypertension in a dosage-dependent manner. Br J Nutr, 2013. 109(12): p. 2247-52.

22. Nilsson, S. C., R. B. Sim, S. M. Lea, V. Fremeaux-Bacchi, and A. M. Blom, Complement factor I in health and disease. Mol Immunol, 2011. 48(14): p. 1611-20.

23. Nicolas, V. and V. Lievin-Le Moal, Antisecretory peptide AF-16 inhibits the Sat toxin-stimulated transcellular and paracellular passages of fluid in cultured human enterocyte-like cells. Infect Immun, 2014.

24. Matson Dzebo, M., A. Reymer, K. Fant, P. Lincoln, B. Norden, and S. Rocha, Enhanced cellular uptake of antisecretory peptide AF-16 through proteoglycan binding. Biochemistry, 2014. 53(41): p. 6566-73.

25. DeMartino, G. N., Purification of PA700, the 19S regulatory complex of the 26S proteasome. Methods Enzymol, 2005. 398: p. 295-306.

1. A kit for detecting proteasome-complement complex-formation in bodily fluids, comprising
a. an antibody specific for a proteasome subunit and
b. an antibody specific for complement factor C3 or complement factor C4.

2. A kit for detecting proteasome-complement complex-formation in bodily fluids, according to claim 1, comprising
a. an antibody specific for proteasome protein AF1 and
b. an antibody specific for complement factor C3c.

3. An immunological assay kit for determining the presence or absence, and/or the concentration, of proteasome-complement-complex-formation in bodily fluids using a first antibody and a second antibody, wherein the first antibody is immobilized on a carrier and the second antibody is modified with a labeling substance, and the first antibody and the second antibody are selected from:
a. an antibody specific for a proteasome subunit and
b. an antibody specific for complement factor C3 or complement factor C4.

4. An immunological assay kit according to claim 3, wherein the first antibody is selected from the group consisting of an antibody specific for proteasome protein AF1, LMP2, 20Salfa6, 20Salfa1,2,3,4 and Rpt5, and wherein the second antibody is selected from the group consisting of an antibody specific for complement factor C3, or an antibody specific for complement factor C4.

5. An immunological assay kit according to claim 3, wherein the first antibody and the second antibody are selected from:
a. an antibody specific for proteasome protein AF1, and
b. an antibody specific for complement factor C3c.

6. An immunological assay kit according to claim 3, which is an ELISA test kit.

7. An immunological assay kit according to claim 4, wherein the antibody specific for proteasome protein AF1 is a monoclonal 3H8 antibody derived from the hybridoma cell culture 2341, which is deposited at the DSMZ under the deposition number DSM ACC3271.

8. An immunological assay kit according to claim 3, wherein the antibody specific for complement factor C3c is a polyclonal antibody specific for complement factor C3c.

9. An immunological assay for determining the presence or absence, and/or the concentration, of proteasome-complement complex-formation in bodily fluids using a first antibody and a second antibody, wherein the first antibody is immobilized on a carrier and the second antibody is modified with a labeling substance, and the first antibody and the second antibody are selected from:
a. an antibody specific for a proteasome subunit and
b. an antibody specific for complement factor C3 or complement factor C4.

10. An immunological assay according to claim 9, wherein the first antibody is selected from the group consisting of an antibody specific for proteasome protein AF1, LMP2, 20Salfa6, 20Salfa1,2,3,4 and Rpt5, and wherein the second antibody is selected from the group consisting of an antibody specific for complement factor C3, or an antibody specific for complement factor C4.

11. An immunological assay according to claim 9, wherein the first antibody and the second antibody are selected from:
a. an antibody specific for proteasome protein AF1 and
b. an antibody specific for complement factor C3c.

12. An immunological assay according to claim 9, which is an ELISA test.

13. An immunological assay according to claim 10, wherein the antibody specific for proteasome protein AF1 is a monoclonal 3H8 antibody derived from the hybridoma cell culture 2341, which is deposited at the DSMZ under the deposition number DSM ACC3271.

14. An immunological assay according to claim 9, wherein the antibody specific for complement factor C3c is a polyclonal antibody specific for complement factor C3c.

15. A method for detecting proteasome-complement complex-formation in bodily fluids, comprising subjecting a bodily fluid to an immunological assay comprising
a. an antibody specific for a proteasome subunit and
b. an antibody specific for complement factor C3.

16. A method according to claim 15, wherein the first antibody is selected from the group consisting of an antibody specific for proteasome protein AF1, LMP2, 20Salfa6, 20Salfa1,2,3,4 and Rpt5, and wherein the second antibody is selected from the group consisting of an antibody specific for complement factor C3, or an antibody specific for complement factor C4.

17. A method for detecting proteasome-complement complex-formation in bodily fluids according to any claim 15, comprising subjecting a bodily fluid to an immunological assay comprising
a. an antibody specific for proteasome protein AF1 and
b. an antibody specific for complement factor C3c.

18. A method for detecting proteasome-complement complex-formation in bodily fluids according to claim 15, wherein the immunological assay is performed as an ELISA test.

19. A method for detecting proteasome-complement complex-formation in bodily fluids according to claim 16, wherein the antibody specific for proteasome protein AF1 is a monoclonal 3H8 antibody derived from the hybridoma cell culture 2341, which is deposited at the DSMZ under the deposition number DSM ACC3271.

20. A method for detecting proteasome-complement complex-formation in bodily fluids according to claim 15, wherein the antibody specific for complement factor C3c is a polyclonal antibody specific for complement factor C3c.

21. A method according to claim 15, wherein the bodily fluid is selected from blood, serum, plasma, milk, tear fluid, tear, semen, seminal fluid, vaginal fluid, saliva, sputum, sweat, ascites, amniotic fluid synovial fluid, gastric fluid, cerebrospinal fluid, spinal fluid, eye fluid, pus and/or mucus.

22. A method according to claim 15, wherein the bodily fluid is from a mammal, selected from the group consisting of a human, a horse, a cow, a pig, a dog, a cat and a camel.

23. A method according to claim 15, wherein the bodily fluid is from a bird.

24-30. (canceled)

31. An immunological assay kit according to claim 4, wherein the antibody specific for complement factor C3 is selected from the group consisting of C3, C3c,C3b, and iC3b, and the antibody specific for complement factor C4 is selected from the group consisting of C4, C4b, iC4b, and C4c.

32. An immunological assay according to claim 10, wherein the antibody specific for complement factor C3 is selected from the group consisting of C3, C3c,C3b, and iC3b, and the antibody specific for complement factor C4 is selected from the group consisting of C4, C4b, iC4b, and C4c.

33. A method according to claim 16, wherein the antibody specific for complement factor C3 is selected from the group consisting of C3, C3c,C3b, and iC3b, and the antibody specific for complement factor C4 is selected from the group consisting of C4, C4b, iC4b, and C4c.
